(19) United States
(12) Patent Application Publication
(10) Pub. No.: US 2016/0030498 A1
(43) Pub. Date: Feb. 4, 2016

(54) HERBAL COMPOSITIONS FOR TREATING PAIN AND SKIN DISORDERS

(71) Applicant: RUPAK ENTERPRISES (P) LIMITED, Madhya Pradesh, Indore (IN)

(72) Inventors: Deepak SHAPETI, Madhya Pradesh, Indore (IN); Madhavi SHAPETI, Madhya Pradesh, Indore (IN)

(73) Assignee: RUPAK ENTERPRISES (P) LIMITED, Madhya Pradesh, Indore (IN)

(21) Appl. No.: 14/784,084

(22) PCT Filed: Apr. 9, 2014
(86) PCT No.: PCT/IN2014/000228

§ 371 Date: Oct. 13, 2015

(30) Foreign Application Priority Data
Apr. 10, 2013 (IN) 1352/MUM/2013

(51) Int. Cl. A61K 36/324; A61K 36/45; A61K 36/736; A61K 36/61; A61K 31/05; A61K 31/22

(52) U.S. Cl. CPC A61K 36/324; A61K 31/05; A61K 31/22; A61K 36/736; A61K 36/61; A61K 36/45

(57) ABSTRACT

The invention relates to herbal compositions having standalone preparations of two phytochemicals, Bakuchiol and Boswellic acid as well as synergistic combinations of Bakuchiol with either Boswellia oil or boswellic acid or both. Bakuchiol in these compositions has assay by HPLC of greater than 50%, whereas boswellic acid is standardized to at least 20% of 3-o-Acetyl-11-keto-beta-boswellic acid. The compositions are either oral—for relief and remission of proliferative skin disorders or topical—for the treatment and management of pain. The invention further provides process of preparation of these compositions. The invention also relates to use of the compositions for treating pain or proliferative skin disorders and various dosage regimens.

### TECHNICAL FIELD

The present invention relates to herbal compositions, comprising Bakuchiol (with total furanocoumarin content less than 100 ppm), a standalone therapeutic agent, optionally with a second therapeutic agent selected from 3-o-Acetyl-11-keto p-Boswellic acid, Capsaicin or any other natural or synthetic NSAID or derivative thereof in a pharmaceutically dermatologically or cosmeceutically, acceptable excipients/carriers, for alleviating, managing, reducing and/or treating acute, sub-acute or chronic musculoskeletal and/or joint pain resulting from a condition derived from an inflammatory disorder and/or vertebrates or subjects genetically predisposed to above-mentioned conditions thereof. All the therapeutic agents used in the present invention may be naturally derived or semi/completely synthetically produced. The present invention further relates to a synergistic herbal composition comprising synergistic therapeutic herbal extract standardized to high purity of Acetyl keto beta boswellic acid (greater than or equal to 20% Acetyl-keto beta boswellic acid by HPLC) isolated from Boswellia serrate, in combination with second therapeutic agent, Bakuchiol with total furanocoumarin content less than 100 ppm isolated from Psoralea corylifolia plant seed powder, for management, treatment, relief and remission of proliferative dermatological conditions, specifically Psoriasis.

### BACKGROUND OF INVENTION

In the general terms, pain can be defined as a physical suffering or discomfort caused by illness or injury. Pain that lasts a long time is called chronic, and pain that resolves quickly is called acute. Although muscle aches and pains may occur due to tension, over-activity or injury, many different conditions affect the muscles and surrounding tissues, including the connective tissues, ligaments and tendons. Joint pain can occur due to injury, and conditions triggering inflammation can also cause joint pain. Because muscles join together at the joints, these two symptoms often occur together. Arthritis can be defined as painful inflammation and stiffness of the joints.

Rheumatoid arthritis (RA) is a long-term disease that leads to inflammation of the joints and surrounding tissues. It can also affect other organs. The cause of RA is unknown. It is an autoimmune disease, which means the body's immune system mistakenly attacks healthy tissue. Infection, genes, and hormone changes may be linked to the disease. RA can occur at any age, but is more common in middle age. Women get RA more often than men. Acute and chronic pain is generally the result of Rheumatoid Arthritis.

Psoriatic Arthritis is a type of inflammatory arthritis that develops in up to 30 percent of people who have the chronic skin condition, psoriasis. Common symptoms of psoriatic arthritis include Pain, swelling, or stiffness in one or more joints, Joints that are red or warm to the touch, Sausage-like swelling in the fingers or toes, known as dactylitis, Pain in and around the feet and ankles, especially tendinitis in the Achilles tendon or Plantar fasciitis in the sole of the foot, changes to the nails, such as pitting or separation from the nail bed, and pain in the area of the Sacrum (the lower back, above the tailbone). The exact causes are not yet known, but a number of genetic associations have been identified in a genome-wide association study of psoriasis and psoriatic arthritis including HLA-B27.

Osteoarthritis (OA) also known as degenerative arthritis or degenerative joint disease or osteoarthrosis, is a group of mechanical abnormalities involving degradation of joints, including articular cartilage and sub-chondral bone. Symptoms may include joint pain, tenderness, stiffness, locking, and sometimes an effusion. A variety of causes such as hereditary, developmental, metabolic, and mechanical may initiate processes leading to loss of cartilage. Short and Long-term pain management and effective treatments generally involve a combination of exercise, lifestyle modification, and analgesics.

Degenerative bone disease is commonly known as degenerative osteoarthritis, caused by inflammation, breakdown and loss of the cartilage of the joints. Sports injuries are those that occur in/during athletic activities. In many cases, these injuries are due to overuse or acute trauma of a part of the body when participating in certain sports activities. Meniscal tears are among the most common knee injuries. Athletes, particularly those who play contact sports, are at risk for meniscal tears. However, anyone, at any age can tear a meniscus.

Baker's Cyst is a benign swelling of the semi-membranous or more rarely, some other synovial bursa found behind the knee joint, often as a result of playing excessive sports.

Tennis/Golf Elbow is a condition where the outer part of the elbow becomes sore and tender, caused by sports such as swimming and climbing and golf, the work of manual workers and waiters, as well as activities of daily living. Repetitive Stress Injury (RSI) is an injury of the musculoskeletal and nervous systems that may be caused by repetitive tasks, forceful exertions, vibrations, mechanical compression (pressing against hard surfaces), sustained, or awkward positions.

Sciatica (sciatic neuritis or lumbar radiculopathy) is a set of symptoms including pain that may be caused by general compression or irritation of one of five spinal nerve roots that give rise to each sciatic nerve, or by compression or irritation of the left or right or both sciatic nerves. Symptoms include lower back pain, buttock pain, and pain, numbness or weakness in various parts of the leg and foot. Other symptoms include pins and needles or tingling and difficulty in moving or controlling the leg. Typically, the symptoms are only felt on one side of the body.

Joint stiffness may be either the symptom of pain on moving a joint, the symptom of loss of range of motion or the physical sign of reduced range of motion. Frozen shoulder, medically referred to as adhesive capsulitis, is a disorder in which the shoulder capsule, the connective tissue surrounding the glenohumeral joint of the shoulder, becomes inflamed and stiff, greatly restricting motion and causing chronic pain.

One or more of these health conditions may occur in vertebrates with active, over-active or unhealthy lifestyles, or those who are genetically predisposed to them, or otherwise.

Treatments have traditionally been in the form of hot/cold packs, topical and oral analgesics, non-steroidal anti-inflammatory drugs (NSAIDs), stronger medications, such as muscle relaxants, anti-anxiety drugs, antidepressants, prescription NSAIDs, steroidal injections at site of joint swelling/inflammation (in case of chronic pain cases), invasive and non-invasive treatments, among others. The main categories of topical analgesics cover:
Rubefacients: traditional formulations based on salicylate and nicotinate esters, capsaicin and capsicum extracts and derivatives.
NSAIDs: diclofenac, felbinac, ibuprofen, ketoprofen, piroxicam, naproxen, flurbiprofen and other NSAIDs.
A miscellaneous group: including benzydamine, mucopolysaccharidepolysulphate, salicylamide and cooling sprays.
Local anesthetics: including lignocaine and benzocaine.

Unfortunately, these non-steroidal anti-inflammatory agents (NSAIDs) have to be taken for long time durations to reduce both the pain and swelling caused by arthritis. Though they are among the most widely used medications, the side effects of these drugs frequently include gastrointestinal (GI) ulceration, indigestion, burning, and bleeding (Wallace and Vong, 2008). Other common side effects associated with the long-term use of NSAIDs include:
indigestion
stomach ulcer (a sore in the lining of the stomach), which in turn may cause:
gastrointestinal bleeding—internal bleeding within the digestive system
anaemia—where blood is unable to carry enough oxygen around your body which can cause shortness of breath and tiredness o gastrointestinal perforation—where a hole occurs in the wall of your stomach or intestines

A class of non-steroidal anti-inflammatory agents (NSAIDs) called COX-2 inhibitors was then identified that could provide significant benefits in the treatment of OA. COX-2 inhibitors were claimed to be devoid of ulcer-promoting effects; however, this promise has been unfulfilled, and there are concerns about the cardiovascular safety of COX-2 inhibitors (Wallace and Vong, 2008; Vardeny and Solomon, 2008). In rare cases, serious stomach problems, such as bleeding, can occur without warning. NSAIDs and COX-2 inhibitors should not be taken by people who are allergic to aspirin (Bavbek et al., 2007; Palmer, 2005).

Another type of medication prescribed to reduce severe/chronic pain and swelling are corticosteroids. Corticosteroid injections offer quick, effective pain relief. However, they can be used “only few times a year because they weaken the bone and cartilage (Silbermann, et al., 1981a; Silbermann, et al. 1981b; Silbermann, et al, 1979; Silbermann et al., 1977). Also, as corticosteroids can cause other potentially serious side effects (Benyamin et al., 2008; Kubota et al., 2008), their use must be monitored by a physician.

Additionally, over the years, and across geographical boundaries, various herbs, herbal extracts, herbal supplements, and powders/oils from herbs have been used for their anti-inflammatory purposes. However, there is no written and experimental evidence of Phytochemicals, specifically in higher Assays by HPLC, being used safely in short and long term management, relief and/or treatment of acute, sub-acute and chronic pain resulting from muscle sprains/strains, inflammation of joints, Arthritis, Rheumatoid Arthritis, Psoriatic Arthritis, Osteoarthritis, Bone degenerative disorders, Meniscal tears (lateral and medial), Baker's cysts (or Popliteal cysts), Tendinitis, Repetitive Stress Injuries (RSI), Sciatica, Joint stiffness, Frozen shoulder, that affect vertebrates, as also human athletes, sportspersons and vertebrates/patients with active, over-active/unhealthy lifestyles, and/or those genetically predisposed to any of the above conditions thereof.

Traditional usage of herbs and extracts, specifically Curcumin has been used as anti-inflammatory agents. However, Curcumin extract, containing total Curcuminoids, Assay by HPLC 95%, is not a single Phytochemical, but a mixture of three and more Phytochemicals. Its low bioavailability and topical penetration issues do not enable effective or long-term management of osteo-arthritis, and bone-degenerative disorders.

Most herbal topical products are rubefactant agents, which as per the Royal Society of Medicine are counter-irritants which work by reddening the skin, by causing blood vessels to dilate, resulting in a feeling of warmth. All these agents are salicylates or derivatives thereof and are present in herbs and herbal extracts of lower/higher Assays by HPLC that have been used for their anti-arthritic properties, comprise White willow bark, Arnica, Devil's claw, camphor menthol, and these primarily show mild analgesic action by acting as a rubefactant and not really reducing localized inflammation.

Therefore, there is still a need in the art to provide an effective, safe and pure herb-derived formulation and/or an Alternative natural product for effective short and long-term management, relief and/or treatment of inflammatory responses ranging from acute, sub-acute to chronic cases, as discussed above, without side-effects arising from NSAIDs. In the light of the above, the objective of the present invention is to provide synergistic herbal compositions, in modified or regular release, topical and/or oral dosage forms, incorporating single or multi-component Phytochemicals, low to high Assays by HPLC, derived from natural sources, one or more herbs/herbal extracts, or semi/completely chemically synthesized, to provide safe and effective solutions for short and long-term management, relief and/or treatment of acute, sub-acute and chronic pain resulting from, but not limited to inflammatory responses, due to, but not necessarily from Arthritis, Rheumatoid Arthritis, Psoriatic Arthritis, Osteoarthritis, Bone degenerative disorders Meniscal tears (lateral and medial), Baker's cysts (or Popliteal cysts), Tendinitis, Repetitive Stress Injuries (RSI), Sciatica, Joint stiffness, Frozen shoulder and injuries that affect vertebrates and human athletes, sportspersons and patients, subjects or vertebrates with active, over-active/unhealthy lifestyles, or/and those genetically predisposed or otherwise to above-mentioned conditions.

Furthermore, anti-inflammatory agents utilized by prior investigators have not incorporated therapeutically-active Phytochemicals, at higher Assays by HPLC, derived herbs/herbal extracts. Thus, one aspect of this invention relates to pain relief and/or management and anti-inflammatory properties of one or more Phytochemicals, derived from one or more herbs, from low to higher Assays by HPLC, incorporated into novel, effective, synergistic herbal compositions, either modified or regular release, topical and/or systemic dosage forms. Phytochemicals with higher Assays by HLPLC imply lesser impurities, greater therapeutic activity and more efficacious properties, and better safety profiles of the active ingredient and the final herbal formulation.

Further, in addition to above mentioned inflammatory responses, it is known that humans and domesticated animals are afflicted with a wide variety proliferative skin disease such as Atopic Dermatitis, Dermatitis, Psoriasis and Eczema. All these disorders are characterized by epidermal cell proliferation, or division, and may also be associated with incomplete tissue differentiation. Proliferative skin disorders are the most serious form of the skin diseases with which this invention is concerned. Additionally, Psoriatic Arthritis is a type of inflammatory arthritis that develops in up to 30 percent of people who have the chronic skin condition, psoriasis.

Psoriasis is a chronic, hereditary disease generally characterized by eruption of erythematous, silvery-scaled plaques, predominantly on the elbows, knees, scalp and trunk, affecting between 1-2% of the population worldwide. Psoriasis is a multifactorial disease of unknown etiology. It has been shown that in some patients alcohol abuse has been associated with psoriasis. Chronic alcohol abuse results in the impairment of health-related, social and occupational functioning. Therefore the association of psoriasis and alcoholism represents one of the major psychodermatological issues where a multidisciplinary approach is crucial for optimal outcome.

Psoriasis is a chronic, inflammatory disease which can affect the skin, joints and nails. In psoriasis, the immune system is mistakenly activated, which leads to overproduction of skin cells. Skin cells build up too rapidly on the surface of the skin, forming raised, red, scaly patches (called plaques). These plaques are often itchy and sometimes painful. Psoriasis lesions commonly appear on the scalp, limbs and lower back, but they can occur anywhere on the body.

Some people are not very affected by their psoriasis symptoms, but for others, psoriasis is a disabling and embarrassing condition that affects their lifestyle and their interactions with others. There are many available treatments that may help to relieve symptoms and improve daily life. Plaque psoriasis is the most common type of psoriasis and is characterised by redness, thickness and scaling. First line management of chronic plaque psoriasis is with topical treatments, including vitamin D analogues, topical corticosteroids, tar-based preparations, dithranol, salicylic acid and topical retinoids.

Recent studies suggest that that psoriasis is not primarily a skin disorder but an immunological disturbance under the skin. The skin manifestations are a result of overstimulation of superficial skin cells (Langerhans cells) due to increased production of interleukin 2, 6 and 8 as well the diminished production of transforming growth-factor-alpha interleukin-10

There are multiple therapeutic options for the treatment of moderate to severe psoriasis. Traditional systemic therapies for psoriasis, including methotrexate (MTX) and cyclosporine (CsA), have a well-documented array of toxicities, particularly end-organ toxicities.

Phototherapy and photochemotherapy are important treatment regimens for inflammatory as well as malignant diseases in dermatology. Both treatment modalities have been developed already three decades ago and therefore profound knowledge exists on the use, efficacy, and long-term side effects. [0028] The typical treatments modalities available to treat for the treatment of proliferative skin diseases include:

(a) topical applications of coal tar derivatives, steroids in high doses
(b) the systemic administration of glucocorticoids
(c) exposure to ultra violet light, x-irradiation, and
(d) in severe cases, surgery is considered as last option.

While these treatments provide, some remission of the original symptoms, however, suffers some defect, for example, temporary and/or incomplete mitigation of symptoms, rapid re-occurrence of the disease when treatment is terminated. Strategies of treatment must address both rapid control of the disease and maintenance of benefits.

Therefore, there is still a need in the art to develop a formulation that can ameliorate one or more aforementioned problems. Herbal formulations are considered to be safe with fewer side effects over its allopathic counter parts, such as topical or systemic steroids. Therefore, further objective of the invention is also to provide a synergistic herbal formulation for relief and remission of proliferative dermatological conditions, specially Psoriasis and its sub-types—Plaque, Pustular, Palmoplantar's psoriasis and Dermatitis and its sub-types—Atopic, Dishydrotic, Urticaria.

### BRIEF DESCRIPTION OF FIGURES

FIG. 1: HPLC of Initial Psoralea corylifolia extract @ 265 nm.

FIG. 2: HPLC of Initial Psoralea corylifolia extract @ 245 nm.

FIG. 3: HPLC of High purity extract of Psoralea corylifolia, standardized to Bakuchiol@ 265 nm (Furanocoumarin content (<100 ppm))

FIG. 4: HPLC of High purity extract of Psoralea corylifolia, standardized to Bakuchiol@ 245 nm (Furanocoumarin content (<100 ppm))

FIG. 5: HPLC graph of initial Boswellia serrata extract, standardized to BOSWELLIC ACID, 20%

FIG. 6: HPLC of high purity 3-o-Acetyl-11-keto P-boswellic acid (BOSWELLIC ACID), 95%

FIG. 7: HPLC of high purity Capsaicin, from a standardized sample of Total Capsaicinoids.

FIG. 8: Provides details on study of treatment and management of pain and inflammation of muscles and joints by standalone Bakuchiol composition.

FIG. 9: Provides a user study for checking efficacy of intervention under example 20.

FIG. 10: provides Psoriasis Severity Score under example 18.

### SUMMARY OF INVENTION

In accordance with the above objective, the invention discloses Bakuchiol (with total furanocoumarin content less than 100 ppm), a standalone therapeutic agent, optionally with a second therapeutic agent selected from 3-o-Acetyl-11-keto p-Boswellic acid, Capsaicin or any other natural or synthetic NSAID or derivative thereof in a pharmaceutically, dermatologically or cosmeceutically, acceptable excipients/carriers, for alleviating, managing, reducing and/or treating acute, sub-acute or chronic musculoskeletal and/or joint pain resulting from a condition derived from an inflammatory disorder and/or vertebrates or subjects genetically predisposed to above-mentioned conditions thereof.

More specifically, present invention provides in a standalone preparation, topical compositions of Bakuchiol for treatment or management of pain.

The present invention also provides synergistic compositions of Bakuchiol and either boswellia oil or boswellic acid standardized to at least 20% 3-o-Acetyl-11-keto p-Boswellic acid for treatment/management of pain and skin disorders such as proliferative dermatological disorders including psoriasis and dermatitis.

Further, present invention also provides standalone preparations of Boswellic acid standardized to at least 20%, preferably at least 30% and most preferably at least 40% of 3-o-Acetyl-11-keto p-Boswellic for the relief and remission of proliferative dermatological conditions.

Further, yet another objective, the present invention further provides a synergistic herbal composition for the for relief and remission of proliferative dermatological conditions comprising in a specific ratio a first therapeutic agent which comprises of a boswellic acid standardized to Acetyl keto beta boswellic acid (greater than or equal to 20% Acetyl keto beta boswellic acid by HPLC) in combination with second therapeutic agent a herbal extract standardized to Bakuchiol with total furanocoumarin content less than 100 ppm. The ratio is from about 30:1 to 1:5 and preferably from 12:1 to 2:1.

The proliferative skin disorders comprise psoriasis, dermatitis, eczema, chronic hyper proliferative dermatological disorders resulting from allergic conditions, some environmental trigger or auto-immune conditions.

More specifically, present invention provides synergistic compositions of Bakuchiol and either boswellia oil or boswellic acid or both for treatment or management of pain or skin disorders. Although the invention is demonstrated with therapeutic ingredients of herbal origin, however, a semi or completely synthetic therapeutic ingredients such as Bakuchiol, 3-o-Acetyl-11-keto p-Boswellic acid or Capsaicin, may be used in the instant invention to achieve substantially similar therapeutic effect. The synthesis of the above therapeutic agents is well known in the art and as such a person skilled in the art can practice the instant invention by using synthetic therapeutic ingredients of the instant invention.

Accordingly, the invention provides synergistic herbal compositions which comprise of a first therapeutic herbal extract, standardized to Bakuchiol (Assays by HPLC between 50-99.99%), with total furanocoumarin content less than 100 ppm, derived from herbs or semi/completely synthesized chemically, and optionally with a synergistic therapeutic agent standardized to 3-o-Acetyl-11-keto-beta-boswellic acid (BOSWELLIC ACID, Assays by HPLC between 20-99%), or optionally with a synergistic therapeutic agent, Capsaicin (Assays by HPLC between 10-99%), latter two either derived from herbs or semi/completely chemically synthesized, or any other naturally-derived and/or chemically synthesized NSA ID or derivative thereof, including but not limited to Ajoene (Allium sativum), Curcuminoids, Zingerberofficinale.

These herbal origin or semi/completely chemically synthesized therapeutic agents are formulated in a suitable vehicle in regular or modified release forms, in topical or oral dosage forms, comprising of excipients, skin penetration enhancers, anti-oxidants, moisturizing agents, emollients, humectants, and any other pharmaceutically, dermatologically, or cosmeceutically acceptable carriers), to form a synergistic herbal composition for short and long-term management, relief and/or treatment of acute, subacute and chronic pain resulting from, but not limited to inflammation due to, but not necessarily from Arthritis, Rheumatoid Arthritis, Psoriatic Arthritis, Osteoarthritis, Bone degenerative disorders Meniscal tears (lateral and medial), Baker's cysts (or Popliteal cysts), Tendinitis, Repetitive Stress Injuries (RSI), Sciatica, Joint stiffness, Frozen shoulder and injuries that affect vertebrates and human athletes, sportspersons and patients, subjects or vertebrates with active, over-active/unhealthy lifestyles, and those genetically predisposed to above-mentioned conditions.

The composition of the invention may be obtained from whole plant or from one or more individual parts and is selected from Psorlea and Boswellia genus, but not limited to other genus like Sassfras, Magnolia, and Astractlydoes.

Thus, according to various aspects herein described, the present invention provides various stand alone (separate) compositions of Bakuchiol and Boswellic acid for the treatment/management of pain for relief/remission in skin disorders such as psoriasis and dermatitis. Boswellia oil is employed in Pain management compositions. Further, according to various aspects herein described the present invention provides various synergistic compositions of Bakuchiol and Boswellia oil/Boswellic acid for the treatment/management of pain and for relief/remission in skin disorders such as psoriasis and dermatitis.
The amount of Bakuchiol in the standalone or synergistic combination compositions is from 1-15% of the composition. The amount of Boswellic acid in standalone or synergistic combination compositions is from 1-90% of the composition.

Further invention provides two types of compositions viz. oral and topical compositions. Preferably, oral compositions are provided for relief and remission of proliferative skin disorders whereas topical compositions are provided for treatment/management of pain. Thus topical composition having Bakuchiol and boswellia oil is preferred for treatment/management of pain whereas oral compositions of Bakuchiol and boswellic acid standardized to at least 20% of 3-o-Acetyl-11-keto p-Boswellic acid is preferred for relief and remission of proliferative skin disorders.

Bakuchiol used in standalone or synergistic composition is standardized to Bakuchiol of assay of at least 50%, preferably 70% or more and most preferably 90% or more. Boswellic acid when used in standalone or synergistic composition is standardized to at least 20%, preferably at least 30% and most preferably at least 40% of 3-o-Acetyl-11-keto p-Boswellic acid.

In several embodiments of oral herbal composition Bakuchiol is present from 10-15%, and Boswellic acid standardized to 3-o-Acetyl-11-keto p-Boswellic acid is present from 30-90% w/w. However when Bakuchiol is present in addition to boswellic acid, it may be present in 1-50% of composition. The embodiments of topical composition has Bakuchiol from 1-15% and Boswellia oil from 0.1-15% of the composition.

### DETAILED DESCRIPTION OF INVENTION

### Definitions

Treatment/management of pain includes short and long-term management, relief and/or treatment of acute, sub-acute and chronic pain resulting from, but not limited to inflammation due to, but not necessarily from Arthritis, Rheumatoid Arthritis, Psoriatic Arthritis, Osteoarthritis, Bone degenerative disorders Meniscal tears (lateral and medial), Baker's cysts (or Popliteal cysts), Tendinitis, Repetitive Stress Injuries (RSI), Sciatica, Joint stiffness, Frozen shoulder and injuries that affect vertebrates and human athletes, sportspersons and patients, subjects or vertebrates with active, over-active/unhealthy lifestyles, and those genetically predisposed to above-mentioned conditions. Skin disorders include dermatological proliferative disease/conditions such as Psoriasis, Atopic Dermatitis, Dermatitis, Eczema, vitiligo, bullous pemphigoid, actinic keratosis, and any other skin disorders.

All the therapeutic agents used in the present invention may be naturally derived or semi/completely synthetically produced. When the therapeutic agents are naturally derived, there is no limitation on any natural species from which they are derived.

The invention will now be described in detail in connection with certain preferred and optional embodiments, so that various aspects thereof may be more fully understood and appreciated.

Present invention provides standalone or synergistic compositions of two therapeutic agents viz. Bakuchiol and Boswellic oil or boswellic acid or combination of the three therapeutic ingredients. Bakuchiol used in standalone or synergistic composition according to present invention is standardized to Bakuchiol of assay of at least 50%, preferably 70% or more and most preferably 90% or more. Bakuchiol is used from 1-15% of the composition. Boswellic acid when used in standalone or synergistic composition according to present invention is standardized to at least 20%, preferably at least 30% and most preferably at least 40% of 3-o-Acetyl-11-keto p-Boswellic acid. Boswellic acid is used from 1-90% of the composition. Boswellia oil is used from 0.1-15% of the composition.

The composition is preferably in the form of oral or topical composition. The oral composition comprises of standalone boswellic acid from 1-90% of composition for treatment of skin disorders. Alternatively oral compositions comprises of synergistic compositions containing boswellic acid from 1-90% of composition and Bakuchiol from 1-15% of the composition for treatment of skin disorders. The ratio of boswellic acid to Bakuchiol in synergistic oral compositions is from 30:1 to 1:5, preferably from 12:1 to 2:1. Generally, oral composition contains boswellic acid whereas topical compositions contain either of boswellic oil or boswellic acid or both. Example 8 Formulation C is representative of composition having Bakuchiol, boswellic acid and Boswellia oil.

The topical compositions for treatment/management of pain are usually in the form of creams, gels, ointments. They may contain standalone Bakuchiol from 1-15% of composition. Alternatively topical compositions may contain synergistic composition containing Bakuchiol from 1-15% of composition and boswellia oil from 0.1 to 15% of the composition. The ratio of Bakuchiol to boswellia oil is from 1:15 to 5:0.1 The composition may also contain boswellic acid from 1-15% of the composition. The ratio of Bakuchiol to boswellic acid in topical composition is from 1:5 to 5:1. The topical composition may also contain combination of the three therapeutic ingredients Bakuchiol, boswellic acid and boswellia oil.

The present invention provides various compositions having one or more therapeutic agent selected from Bakuchiol and Boswellia oil/Boswelllic acid for the treatment/management of pain or for skin disorders.

Accordingly, in a preferred embodiment, the invention discloses herbal compositions comprising a first therapeutic herbal extract standardized to Bakuchiol (Assays by HPLC between 50-99.99%) with total furanocoumarin content less than 100 ppm, derived from herbs or semi/completely chemically synthesized, optionally with a synergistic second therapeutic agent which is boswellia oil/boswellic acid (boswellia extract) standardized to 3-o-Acetyl-11-keto β-boswellic acid (BOSWELLIC ACID) (Assays by HPLC between 20-99%), further optionally in combination with Capsaicin (Assays by HPLC between 10 to 99%) derived from herbs or semi/completely chemically synthesized, or any other naturally-derived, low to high Assays by HPLC, and/or semi/completely chemically synthesized NSAID or derivative thereof, including but not limited to Ajoene (from Allium sativum), Curcuminoids, Zingiberofficinale. These above-mentioned herb-derived and/or semi/completely chemically synthesized therapeutic agents are formulated in regular or modified release, topical or systemic dosage forms, in suitable vehicle(s) comprising of excipients, skin penetration enhancers, anti-oxidants, moisturizing agents, emollients, humectants, and any other pharmaceutically dermatologically and/or cosmeceutically acceptable carrier(s).

Although, all the above active ingredients have been described in prior art individually as broadly having anti-inflammatory compounds, however, none of the prior art indicates specific activity in-vivo for that therapeutic area, attributable to the use of individual mono-component phytochemicals in low or high Assays by HPLC, or a specific synergistic herbal./semi-synthetic combination of these together (multi-component Phytochemicals and/or herbal extracts) and the safety gains involved in the use of these standardized finished herbal compositions incorporating these therapeutic agent/agents, with higher Assays by HPLC. The synergistic action and excellent safety profile of this preferred embodiment make these compositions of matter a superior alternative both in terms of safety and efficacy over the existing known rubefactant compositions or other low assays/non-standardized herb-derived or semi/completely chemically synthesized NSAID compositions.

The amount of Bakuchiol in one of the embodiments of herbal formulation as a stand-alone therapeutic agent, is in the range of 1-15% w/w of total composition, or optionally with 3-o-Acetyl-11-keto p-boswellic acid (BOSWELLIC ACID) in the range of 1-15% w/w of total composition, or optionally with Capsaicin, in the range of 0.1-5% w/w of total composition. In the preferred embodiment, the Assay by HPLC percent of Bakuchiol is at least 50% and that of 3-o-Acetyl-11-keto P-boswellic acid (BOSWELLIC ACID) is from at least 20% HPLC. In the preferred embodiment, the Assays by HPLC percents of Bakuchiol is at least 50% and that of Capsaicin is in the range of 10-99% by HPLC.

In a preferred embodiment, the composition comprises standalone therapeutic agent, standardized to Bakuchiol (Assay by HPLC at 99%), with total furanocoumarin content less than 100 ppm in an amount of 2% w/w of total composition in suitable pharmacologically, cosmetically and/or dermatologically acceptable carriers, as provided in Example 8, Formulation A.

In another preferred embodiment, the composition comprises first therapeutic agent, standardized to Bakuchiol (Assay by HPLC 99%), with total furanocoumarin content less than 100 ppm, in an amount of 1.5% w/w of total composition, and second therapeutic agent Boswellia oil in an amount of 4.9% of the total composition in suitable pharmacologically, cosmetically and/or dermatologically acceptable carriers, as provided in Example 8, Formulation B.

In yet another preferred embodiment, the composition comprises first therapeutic agent, standardized to Bakuchiol, (Assay by HPLC 99%) with total furanocoumarin content less than 100 ppm, in an amount of 2.5% w/w of total composition and a second therapeutic agent, standardized to 3-o-Acetyl-11-keto-p-boswellic acid (BOSWELLIC ACID, Assay by HPLC 20%) in an amount of 1% w/w of total formulated composition and third therapeutic agent Boswellia oil in an amount of 8% of the total composition as provided in Example 8, Formulation C.

In a further embodiment, the composition comprises a first therapeutic agent, standardized to Bakuchiol (Assay by HPLC 99%), with total furanocoumarin content less than 100 ppm, in an amount of 4% w/w of total composition and a second therapeutic agent, standardized to Capsaicin (Assay by HPLC 96%) in an amount of 0.5% w/w of total composition and third, Boswellia oil in 0.1% of total composition as provided in Example 8, Formulation D.

The preferred embodiment of the invention provides substantial and quantifiable improvement in pain indices in vertebrates. In one embodiment, the present disclosure provides a composition comprising as stand-alone herbal composition of Bakuchiol with low level of furanocoumarin impurities, optionally with 3-o-Acetyl-11-keto β-boswellic acid (BOSWELLIC ACID), and/or optionally with Capsaicin or any other naturally-derived or semi/completely chemically synthesized NSAID/anti-inflammatory agent or derivative thereof.

In yet another embodiment, the invention provides a method for treating or alleviating and management of acute pain resulting from Arthritis, Rheumatoid Arthritis, Psoriatic Arthritis, Osteoarthritis, Bone degenerative disorders Meniscal tears (lateral and medial), Baker's cysts (or Popliteal cysts), Tendinitis, Repetitive Stress Injuries (RSI), Sciatica, Joint stiffness, Frozen shoulder, by administering the standardized herbal composition comprising a stand-alone therapeutic agent, standardized to Bakuchiol with low level of furanocoumarin impurities, or optionally in combination with a second therapeutic agent, standardized to 3-o-Acetyl-11-keto β-boswellic acid (BOSWELLIC ACID) or optionally in combination with another therapeutic agent, standardized to Capsaicin, or any other naturally-derived or semi/completely-chemically synthesized NSAID or derivative thereof.

In yet another embodiment, the invention provides a method for reducing the intensity of chronic or acute pain, tenderness, swelling or restricted mobility, wherein the method comprises of administration of efficacious amount of the herbal composition comprising a stand-alone therapeutic agent, standardized to Bakuchiol with low level of furanocoumarin impurities, or optionally in combination with a second therapeutic agent, standardized to 3-o-Acetyl-11-keto p-boswellic acid (BOSWELLIC ACID) or optionally in combination with another therapeutic agent, standardized to Capsaicin, or any other naturally-derived or semi/completely-chemically synthesized NSAID/anti-inflammatory agent or derivative thereof.

The method according to the invention, wherein the acute, sub-acute or chronic condition is resulting from but not limited to sports/sporting injuries, repetitive stress injuries, or bone and joint degenerative disorders, or age-related bone, muscle or joint disorders, or those with active, over-active/unhealthy lifestyles, or vertebrates genetically predisposed to inflammation of joints and/or muscles.

The compositions of the invention are formulated modified or regular release, topical and/or systemic dosage forms, in suitable vehicles, comprising of pharmaceutical inert ingredients, excipients, diluents, binders, fillers, preservatives, emollients, penetrating enhancers, solubilizers, moisturizers, such as but not limited to cyclodextrin, methyl cellulose, paraffins, castor oil, almond oil, vitamin E, methyl paraben, propyl paraben and the like, into an systemic dosage form including but not limited to tablet, soft-gel, capsule, nasal drops/sprays, inhaler, injections, or a topical dosage form including but not limited to ointments, gels, emulgels, nano-emulgels, lotions, creams, salves, balms, liniments, oils, patches and the like, by using the methods known in the art.

For example, the disclosed method reduces intensity of pain and range of functioning in cases of inflammatory responses due to, but not limited to Meniscal tears, Sciatica, Joint stiffness, Frozen shoulder and Bakers cyst.

In certain embodiments the pain may be acute, sub-acute or chronic resulting from inflammatory responses, due to but not limited to repetitive stress injuries or from degenerative disorders likes arthritis, rheumatoid arthritis, psoriatic arthritis and osteoarthritis. The invention includes the formulation of Bakuchiol as stand-alone active ingredient, from low to higher Assays by HPLC (%), with furanocoumarin content less than 100 ppm, optionally with second therapeutic agent, standardized to 3-o-Acetyl-11-keto p-Boswellic acid (BOSWELLIC ACID), from low to higher Assays by HPLC (%), optionally with another therapeutic agent, standardized to Capsaicin, in cosmetic/dermatologically/pharmaceutically acceptable base for obtaining a cream, gel, emulgel nano-emugel, lotion, salve, balm, ointment, Liniment, oil or patch, which is to be applied topically, on and around the site of pain.

The present provides herbal compositions of Bakuchiol and combination of Bakuchiol with other phytochemicals, mainly boswellia oil/boswellic acid (boswellic extract) or combination of all the three therapeutic agents to treat variety of painful conditions such as for short and long-term management, relief and/or treatment of acute, sub-acute and chronic pain resulting from, but not limited to inflammation due to, but not necessarily from Arthritis, Rheumatoid Arthritis, Psoriatic Arthritis, Osteoarthritis, Bone degenerative disorders Meniscal tears (lateral and medial), Baker's cysts (or Popliteal cysts), Tendinitis, Repetitive Stress Injuries (RSI), Sciatica, Joint stiffness, Frozen shoulder and injuries that affect vertebrates and human athletes, sportspersons and patients, subjects or vertebrates with active, over-active/unhealthy lifestyles, and those genetically predisposed to above-mentioned conditions.

The present invention also provides herbal compositions containing Bakuchiol in combination with boswellic acid standardized to 3-o-Acetyl-11-keto-beta-boswellic acid (BOSWELLIC ACID, Assays by HPLC between 20-99%), to treat skin disorders such as psoriasis, dermatitis, eczema, chronic hyper proliferative dermatological disorders resulting from allergic conditions, some environmental trigger or auto-immune conditions.

The inventors of the present invention have surprisingly found that herbal compositions of phytochemical Bakuchiol herein described wherein Bakuchiol has been standardized to an assay of at least 50%, more preferably at least 70% and most preferably at least 80% in a topical composition provides effective pain management. Without limitations, representative compositions are provided under example 8(A). These compositions may further contain boswellia oil, an essential oil with Anti-inflammatory and analgesic activity, which provides anti-inflammatory, soothing action and enhances effect of standalone Bakuchiol compositions synergistically. Without limitations, representative compositions comprising combination of Bakuchiol and boswellia oil are provided under example 8 (B). Further the compositions may also contain boswellic acid standardized to at least 20%, preferably 30% and most preferably 40% of 3-o-Acetyl-11-keto-beta-boswellic acid. The representative compositions are described under example 8 Formulation C.

Further, inventors have found that herbal compositions of phytochemical Bakuchiol herein described wherein Bakuchiol has been standardized to an assay of at least 50%, more preferably at least 70% and most preferably at least 80% and boswellic acid standardized to 3-o-Acetyl-11-keto-beta-boswellic acid (BOSWELLIC ACID, Assays by HPLC at least 20%), provide surprisingly better therapy in various skin disorders such as psoriasis, dermatitis, pruritis and eczema and other proliferative skin disorders. Without limitations, representative compositions are provided under example 10, 11, 12 and 13.

The herbal compositions of present inventions are preferably topical or oral. Topical preparations cover creams, gels, ointments, lotions, liniments etc. The oral preparations cover tablets, capsules, syrups and likes. Topical compositions of present invention are preferred in pain management whereas oral formulations are preferred in psoriasis and other skin disorders. In topical preparations for pain management, various oils, humectant and emollients are added which further sooth the pain afflicted body part. However in skin treatment, particularly in skin proliferative diseases, skin may have become sensitive to ingredients which are otherwise acceptable in a topical preparation. Some oils may provide allergic responses. However it should be noted that the herbal compositions of the two phytochemicals in topical form by carefully selecting topical ingredients described herein to avoid any allergic response or hypersensitivity response, can be used for skin disorders such as psoriasis, ezcema, pruritis, dermatitis and other disorders. Similarly, oral compositions of the two phytochemicals can be used for pain management. However preferably, we recommend use of topical preparations for pain management and oral preparations for skin disorders. One reason is that oral treatment for psoriasis is shorter than the treatment for pain. Hence for skin disorders we recommend to take orally the combination of Bakuchiol and Boswellic acid for a period of 2-3 months, using the same or even higher dosage for longer periods such as 6 months or more may have some side effects. Topical cream is designed in such a way that it would allow only required amount of therapeutic agents to penetrate dermal layers and can be used for such longer periods without safety concern.

Whether it is for pain management or treatment for proliferative skin disorders such as psoriasis, following factors play a role when Bakuchiol and boswellia oil/boswellic acid are used in standalone preparations or Bakuchiol and boswellic oil/boswellic acid or all three are used in combination to act synergistically.

1) Amounts (doses) and ratio of two phytochemicals selected
2) Assay/content of phytochemicals
A third factor which is more important for treatment and management of skin disorders such as psoriasis, dermatitis and pruritis is treatment or dosing regimen,
3) Treatment or dosing regimen
4) Additionally, composition characteristics are essential to achieve and enhance the action of phytochemical therapeutic agents.

### Amounts (Doses) and Ratio of Two Phytochemicals Selected

The doses of Bakuchiol and boswellia oil/boswellic acid by oral route and topical route need not be the same. The doses usually depend upon two factors 1) type of treatment and 2) route of administration.

In a topical composition for management of pain, primary active agent is Bakuchiol which can be used alone or in combination with boswellia oil or boswellic acid or both. Boswellia oil which does not contain any 3-O-Acetyl-11-keto beta boswellic acid, but contains other anti-inflammatory medium to low molecular weight aldehydes. Preferably Bakuchiol is used in amounts 1-15%, most preferably Bakuchiol is used in amounts 1-5%. When boswellic oil is used along with Bakuchiol, it is used in 0.1-15% of the composition, most preferably in 0.1-5% of the composition. Ratio of Bakuchiol to Boswellia oil is from 1:15 to 5:0.1. When both Bakuchiol and boswellic acid are used in a topical composition, the ratio of Bakuchiol to boswellic acid is from 15:1 to 1:15, preferred ratio is from 10:1 to 1:10 and most preferred is 5:1 to 1:5.

In one embodiment, Bakuchiol is used in 2% of total composition and boswellic oil is used in amounts of 2.5% of the composition. In another embodiment, Bakuchiol is used in 1.5% and boswellic oil is used around 5% of the composition. When combination of boswellic acid and Bakuchiol is used in oral composition to treat psoriasis, the ratio is from 30:1 to 1:5, preferably from 15:1 to 1:2 and most preferably around 12:1 to 2:1.

In oral compositions, when Bakuchiol is present in addition to boswellic acid, it may be present from 1-50% of the composition. In one embodiment, oral capsule contains 175 mg of boswellic acid and 25 mg of Bakuchiol. In yet another embodiment, oral capsule contains 225 mg boswellic acid and 50 mg Bakuchiol. For psoriasis, oral dose of boswellic acid is from 50-500 mg, preferably from 100-400 mg and most preferably from 150-250 mg per unit dosage form. For psoriasis, when Bakuchiol is combined with boswellic acid, usual dose is from 10-100 mg, preferably from 20-50 mg and most preferably from 20-30 mg. For management of pain, the topical composition preferably contains 1-15% of Bakuchiol, most preferably around 1-5% of Bakuchiol. Usually, topical compositions are available in 15 g, 30 g, 45 and 60 g tubes. A topical composition of 30 g having 2% Bakuchiol has 0.6 g of Bakuchiol (Assay≧than 70 w/w) which is used over 2-3 weeks' time and hence providing daily dose of 20-50 mg of Bakuchiol. A topical composition of 30 g having 5% Bakuchiol which is used over 2-3 weeks' time provides higher daily doses from 70-110 mg.

### Assay/Content of Phytochemicals

In a topical preparation, Bakuchiol or babchi oil with assay around 40-70% showed therapeutic effect however did not produce a cream of good consistency and functional characteristics. Hence assay should be 50% and above preferably above 70%, most preferably above 80%. Assay of Bakuchiol above 90% is most desirable. In oral preparation, the therapeutic effect is observed when Bakuchiol of 70% assay is used but the effect was static, hence an assay of greater than 70% more preferably greater than 80% and most preferably 90% and above is desirable. In oral preparation, assay of boswellic acid should be greater than 20%, preferably greater than 30% and most preferably >40%.

### Treatment Regimens

### For Pain Management

In another embodiment, the composition of the invention substantially reduces the pain and inflammation of muscles and joints. Application of topical composition, comprising stand-alone therapeutic agent, standardized to Bakuchiol, 2% w/w of total composition, formulated in a cream base (as per Example 8, Formulation A) and a human pilot study was carried out on subjects suffering from inflammatory responses resulting from acute and sub-acute injuries, due to sporting injuries, repetitive stress injuries, and degenerative disorders, demonstrated excellent efficacy and safety profile with majority of the subjects showing substantial reduction in pain and quality of life (QOL) parameters improvement. All subjects were monitored by analysis of changes in various parameters by Wilcox Signed rank test. The results did not show statistical significance in terms of improvement with Formulation A, and it indicates that Bakuchiol is the primary active ingredient herein. As shown in table 1, all subjects were monitored by statistical analysis of changes in various parameters by Wilcox Signed Rank test. The results indicated that all subjects experienced significant reduction in pain, joint tenderness, joint swelling, mobility restriction, and early morning joint stiffness.

TABLE 1
Statistical analysis of changes in various parameters

| Symptomatic | Mean score | | |
|---|---|---|---|
| relief | Before Treatment | After Treatment | p value |
| Pain intensity | 2.47 ± 0.59 | 1.01 ± 0.41 | p < 0.0001 |
| Swelling | 2.04 ± 0.37 | 1.41 ± 0.38 | p < 0.0001 |
| Tenderness | 2.29 ± 0.49 | 1.37 ± 0.53 | p < 0.0001 |
| Movement | 2.61 ± 0.58 | 1.18 ± 0.67 | p < 0.0001 |

TABLE 2
Effect on Joint Pain Intensity

| Parameter | Baseline | 3 Days | 7 Days | 30 Days | 60 Days |
|---|---|---|---|---|---|
| Mean | 2.47 | 1.39 | 1.06 | 0.67 | 0.41 |
| StdDev | 0.58 | 0.61 | 0.72 | 0.77 | 0.64 |
| Std Error | 0.08 | 0.09 | 0.10 | 0.11 | 0.09 |
| Confidence | 0.21 | 0.22 | 0.26 | 0.28 | 0.24 |
| Interval | | | | | |
| Lower CI | 2.26 | 1.16 | 0.80 | 0.39 | 0.17 |
| Upper CI | 2.68 | 1.61 | 1.33 | 0.96 | 0.64 |

| | |
|---|---|
| Annova | F = 59.1659889094271 |
| | p < 0.0001; HS |

TABLE 3
Effect on Joint Swelling

| Parameter | Baseline | 3 Days | 7 Days | 30 Days | 60 Days |
|---|---|---|---|---|---|
| Mean | 1.94 | 1.39 | 1.10 | 0.90 | 0.65 |
| StdDev | 0.32 | 0.57 | 0.62 | 0.68 | 0.66 |
| Std Error | 0.05 | 0.08 | 0.09 | 0.10 | 0.09 |
| Confidence | 0.12 | 0.21 | 0.23 | 0.25 | 0.24 |
| Intervals | | | | | |
| Lower CI | 1.82 | 1.18 | 0.87 | 0.65 | 0.41 |
| Upper CI | 2.06 | 1.60 | 1.33 | 1.15 | 0.90 |

| | |
|---|---|
| Annova | F = 34.9436201780413 |
| | p < 0.0001; HS |

TABLE 4
Effect on Joint Tenderness

| Parameter | Baseline | 3 Days | 7 Days | 30 Days | 60 Days |
|---|---|---|---|---|---|
| Mean | 2.08 | 1.39 | 1.20 | 0.84 | 0.49 |
| StdDev | 0.45 | 0.57 | 0.61 | 0.69 | 0.71 |
| Std Error | 0.06 | 0.08 | 0.09 | 0.10 | 0.10 |
| Confidence | 0.17 | 0.21 | 0.23 | 0.25 | 0.26 |
| Interval | | | | | |
| Lower CI | 1.92 | 1.18 | 0.98 | 0.58 | 0.23 |
| Upper CI | 2.25 | 1.60 | 1.43 | 1.09 | 0.75 |

| | |
|---|---|
| Annova | F = 47.2235294117644 |
| | p < 0.0001; HS |

TABLE 5
Effect on Early Morning Joint Stiffness

| Parameter | Baseline | 3 Days | 7 Days | 30 Days | 60 Days |
|---|---|---|---|---|---|
| Mean | 2.55 | 1.67 | 1.16 | 0.59 | 0.53 |
| StdDev | 0.50 | 0.52 | 0.51 | 0.61 | 0.58 |
| Std Error | 0.07 | 0.07 | 0.07 | 0.09 | 0.08 |
| Confidence | 0.18 | 0.19 | 0.19 | 0.22 | 0.21 |
| Interval | | | | | |
| Lower CI | 2.37 | 1.48 | 0.97 | 0.37 | 0.32 |
| Upper CI | 2.74 | 1.86 | 1.35 | 0.82 | 0.74 |

| | |
|---|---|
| Annova | F = 115.603418803419 |
| | p < 0.0001; HS |

Conclusion—The composition according to present invention can be effectively used for pain management over extended period of time without any side effects and are efficacious demonstrated by the significant reduction in pain intensity and other symptomatic reliefs.
Dose for pain management—Topical composition for example, cream containing Bakuchiol or combination of Bakuchiol and boswellia oil should be applied topically to the affected body part whenever required. Recommended use is thrice a day and amount applied per application is around 0.5-1 g which however again depends on and vary with the surface area of the body part affected by pain. The topical compositions are safe and can be used continuously over extended period such as 3 months, 6 months and even an year.

### Skin Disorders

This is most crucial part of therapy for psoriasis, dermatitis and other skin disorders.

### Psoriasis

For psoriasis, desired outcome of the treatment is
1. Continued Remission and symptomatic relief
2. No minor or major side effects
3. Reduction in Psoriasis intensity score if PASI score is high
4. No relapse
Two weeks treatment with oral composition having 157 mg of BOSWELLIC ACID and 100 mg of Bakuchiol on a daily dosage basis provided symptomatic relief and remission. This relief was seen as a halting the progression of the lesions and disease condition, but not reversing or improving the condition.
Hence another regimen was adopted. Two×4 weeks regimen during which the oral composition is given twice a day thus making it a continuous 8 weeks regimen provided following effects in various types of psoriasis such as inverse, plaque and pustular.
1. There was continuous remission, no relapse even when a treatment is stopped after 8 weeks regimen and six months after that are over
2. No side effects
3. Significant reduction in Psoriasis score
Dose—Hence, 8 weeks regimen with oral capsule to be taken twice a day having composition containing combination of Boswellic acid and Bakuchiol is recommended.
Unresponsive psoriasis—Our target was to treat and manage cases of Psoriasis which were unresponsive to steroidal medication and other therapeutic standards of care. Specifically, cases of palmoplantar psoriasis were targeted as they respond poorly to medication and restrict patient lifestyle severely due to being in high impact and load bearing areas of the body. An optimized phytotherapy treatment regimen of 22 days, twice a day oral tablets/capsules was created, which led to remission of psoriatic pustules, plaques, and dry scaly skin. This was by replacing the standard of care; corticosteroids like Clobetasol and Halobetasol, and methotrexate
The product is especially relevant in cases of substitution of patients being treated with methotrexate, who have they had a familial history of fibrosis and other autoimmune disorders, which makes a methotrexate standard of care regimen a high risk one.
Dose—The treatment regimen of 22 days, b.i.d, 400 mg capsules, was found to provide remission (85% reduction in Psoriasis scores, scores>5.6<36.4) in mild to moderate cases of plaque psoriasis, pustular, and plamoplantar psoriasis.
The results of user study are included in example 18.

### Dermatitis

Treatment Regimen for Dermatitis—

For dermatitis, desired outcome of the treatment is
1. Continued Remission and symptomatic relief
2. No minor or major side effects
3. Significant reduction in PO-SCORAD
4. No relapse
A treatment for dermatitis 84 weeks regimen with oral capsule to be taken once/twice a day having composition containing combination of boswellic acid and Bakuchiol is recommended. In some mild cases, maintenance by just applying moisturizer would be sufficient whereas in other cases, such treatment should be continued till 8 week time.

### Characteristics of Compositions

Topical compositions—Topical composition according to present invention is preferably cream. Topical cream composition according to the present invention comprises one or more active ingredients, aqueous phase, and a cream base. An aqueous phase comprises mainly water and water soluble/miscible ingredients. Aqueous phase may also contain other aqueous vehicles such as rose water, This phase may contain humectant such as glycerin and propylene glycol. It may contain water soluble preservatives. Aqueous phase is around 55±10% of the total composition. The cream base is around 45±10% of the total composition and includes oils and waxes. The cream base includes oils, penetration enhancers, emollients, moisturizing agents, oil soluble preservatives etc. Waxes include emulsifying agents such as cetyl alcohol, cetostearyl alcohol, beeswax, emulsifying wax, glyceryl monostearates etc.

The actives include Bakuchiol and other actives such as Boswillic oil. Boswellic oil when added along with Bakuchiol, it enhances the activity of Bakuchiol. It is an essential oil with Anti-inflammatory/analgesic activity. It has soothing, calming effect on aching joints, back and arthritic pain. Some oils though not actives often contribute to the enhanced effect in pain management. Eucalyptus/Nilgiri is added to enhance anti-inflammatory action of one or more actives. Almond oil is also added which has multiple role to play. It has emollient action, it relives pain and stress of muscles and also useful for the treatment of chronic dry skin conditions such as eczema or psoriasis. Yet oil is Wintergreen oil which is helpful in muscle and joint discomfort, arthritis and psoriasis. Other oils include emollients and moisturizing agents such as dimethicone and vitamin E oil, each up to 5%. Yet other oils include cedar wood oil which has sedating action and relieves from itchy skin. It is also helpful in psoriasis.

The penetration enhancer is selected from the group consisting of Oleic Acid, Lauric Acid, Isopropyl myristate, Isopropyl Palmitate, Lauryl Alcohol, Azone, Cyclodextrins and derivatives, Pyrrolidones, Tween 80, Sodium hylanurate, Ascorbate, Urea, LechithinPropylene Glycol, Terpenes, jojoba oil, castor oil and methyl sulfonyl methane or any combinations thereof. The amount of penetration enhancer is crucial and helps in penetration of actives through skin layers to produce pain relief. The amount of penetration enhancer should be at least 5%, preferably at least 10% and most preferably at least 15% but usually not more than 20%. It is understood that when combination of penetration enhancers is used, the amount required is applicable to the total of all penetration enhancers.

In one embodiment, combination of 10% of jojoba oil and 8% castor oil is used as penetration enhancer. In one more embodiment, 5% jojoba oil, 3% castor oil and 2% methyl sulphonyl methane is used. In yet another embodiment 10% castor oil and 5% jojoba oil is used. Oil soluble preservatives include methyl and propyl parabens. Alternatively, salts of the parabens can be used as preservatives and added in aqueous phase. Cedar wood oil is also an oil soluble preservative.

The cream base comprises of waxes selected from one or more of cetyl alcohol, cetostearyl alcohols, emulsifying wax and bees wax, glycerylmonostearate. These waxes act as emulsifying agents and help in cream preparation and enhance texture of cream. Emulsifying wax can be added up to 10%, most preferably up to 7%. Glycerylmonostearate is added from 5-10%, most preferably at least 6% and above. Cetyl alcohol and cetostearyl alcohol may be added each up to 2% of the composition.

The above topical composition is most suitable for the management of pain. Some ingredients like Eucalyptus oil and oil of wintergreen which otherwise provide anti-inflammatory action on normal intact skin might produce burning sensation to already inflamed and sensitive skin. Hence such ingredients can be removed for making topical composition for psoriasis.

The following examples, which include preferred embodiments, will serve to illustrate the practice of this invention, it being understood that the particulars shown are by way of example and for purpose of illustrative discussion of preferred embodiments of the invention.

### Preparation of Topical Compositions

The topical compositions according to present invention are prepared as follows:
1. Oily phase ingredients are weighed and added to a vessel
2. Aqueous phase ingredients are weighted and added to another vessel
3. Both the phases are heated on water bath separately at temperature from 70° C. to 90° C.
4. Complete melting/dissolution of all ingredients is ensured by confirming clarity of both the phases
5. Aqueous phase is added into oily phase and mixed with constant stirring for 15 mins
6. When mixture is thick/semisolid, it is transferred to tube filling machine

### Oral Compositions

Oral compositions comprising boswellic acid as standalone therapeutic agent or combination of boswellic acid and Bakuchiol comprise of one or more of diluents, binders, disintegrants, lubricants, glidants and agents that enhance bioavailability of therapeutic agents. Diluents are selected from lactose, microcrystalline cellulose, mannitol, sorbitol, magnesium carbonate, cyclodextrins, yellow dextrin and combinations thereof. Binders are selected from hydroxyl propyl cellulose, hydroxypropylmethylcellulose, polyvinyl pyrrolidones, gum acacia, guar gum, tragacanth and sodium alginate. Disintegrants are selected from the group consisting of microcrystalline cellulose, corn starch, bentonite, sodium starch glycolate, croscarmellose sodium and crospovidone. Lubricants and glidants are selected from magnesium stearate, calcium stearate, colloidal silicon dioxide etc. Oral compositions include capsules, tablets, syrups and likes. Syrup may contain sweeteners and taste masking agents, viscosity building agents, antioxidants, preservatives and buffers.

In another embodiment, the invention provides method of Application of the composition. In accordance with this method, the effect of this composition can be maximized by the following methods of application, by applying an effective amount of the preferred embodiment on the exact location or area around the site of the pain, and massaging the preferred embodiment on the skin. The composition in such case may be selected from the group consisting of, but not limited to creams, salves, ointments, lotions, oils, gels or patches. Alternately, the preferred embodiment of the invention may be systemically applied/administered by way of including but not limited to spraying, inhaling, and ingestion in form of capsules, syrups, tablets, throat drops, pastes or powders. Furthermore, repeated application of the above compositions periodically ensures longer pain free durations. Furthermore, the subject of this invention is non-oily, non-sticky, and does not leave any visible residue or stains. The composition of the preferred embodiment allows for daily and frequent use and higher patient compliance with substantial improvement of quality of life for acute, sub-acute and/or chronic pain-affected patients/vertebrates. There are many herbs reported in Ayurveda and other alternative medicines known to work as anti-inflammatory agents.

The inventors of the present invention propose that the above-mentioned synergistic herbal composition(s), in modified or regular release, topical and/or systemic dosage forms, comprising stand-alone Phytochemical, standardized to Bakuchiol, with furanocoumarin content less than 100 ppm, or optionally in combination with other Phytochemicals, including by not limited to those standardized to 3-o-Acetyl-11-keto p-boswellic acid (BOSWELLIC ACID), Capsaicin, in low to high Assays by HPLC, is effective in short and long term pain management, treatment resulting from, but not limited to inflammation due to, but not necessarily from Arthritis, Rheumatoid Arthritis, Psoriatic Arthritis, Osteoarthritis, Bone degenerative disorders Meniscal tears (lateral and medial), Baker's cysts (or Popliteal cysts), Tendinitis, Repetitive Stress Injuries (RSI), Sciatica, Joint stiffness, Frozen shoulder and injuries that affect vertebrates and human athletes, sportspersons and patients/vertebrates with active, over-active/unhealthy lifestyles, and those genetically predisposed or otherwise, to above-mentioned conditions, to the best of their knowledge, as safe and efficacious standardized herbal/Alternative remedy.

According to a further preferred embodiment, the present invention provides a synergistic herbal composition comprising a first therapeutic herbal extract standardized to Bakuchiol with total furanocoumarin content less than 100 ppm isolated from Psoralea-corylifolia in combination with second therapeutic agent which comprises of a synergistic herbal extract standardized for high purity of Acetyl keto beta boswellic acid (greater than or equal to 20% Acetyl keto beta boswellic acid by HPLC), optionally in association with one or more excipients, for relief and remission of proliferative dermatological conditions. Bakuchiol is a natural phenol and a meroterpene (a chemical compound having a partial terpenoid structure, found in Psoralea-corylifolia and in Otholobiumpubescens. Bakuchiol as used in the instant invention is isolated from the hexane extract of Psoralea-corylifolia plant seed powder. Acetyl keto beta boswellic acid also known as Acetyl-11-keto-beta-boswellic acid (BOSWELLIC ACID) is a naturally occurring pentacyclic triterpene isolated from the methanolic extract of gum resin exudate from the stem of the tree Boswellia serrata (frankincense), for the purpose of present invention.

In one embodiment, the present invention provides a composition comprising Bakuchiol with low level of furanocoumarin impurities, and 3-o-Acetyl keto beta boswellic acid. The composition is isolated from plants.

Another embodiment of the present invention refers to method of use of a composition comprising of Acetyl-11-keto-beta-boswellic acid (BOSWELLIC ACID) and Bakuchiol for alleviation and management of skin rashes, itching, and boils resulting from skin proliferative disorders, specifically psoriasis, atopic dermatitis. For example, the method of use disclosed herein may be administered alongside steroidal therapy during the steroid dose tapering phase, followed by standalone administration of the combination provides complete remission of skin conditions, and is maintained that upon continuance of recommended dosage. In certain embodiments the skin disorder may be resulting from irritants, allergens, trigger, or autoimmune reasons, and the combination defined herein is effective in all of the above. The present invention includes the formulation of a Bakuchiol and BOSWELLIC ACID combination in an oral dosage form or, lotion, cream, ointment which can be applied topically. A dosing regimen of a once daily capsule alone, or application of cream in human subjects suffering from dermatological disorders is demonstrated excellent efficacy and safety profile with all subjects showing complete remission in case of the former, and majority of the subjects showing substantial remission for the latter. Parameters such as itchy sensation, redness were also reduced. In one embodiment, a composition comprising 13.39% (87% assay) and 44.64% 3-o-Acetyl-11-keto-beta-boswellic acid (BOSWELLIC ACID) (33% assay) formulated in a capsule and a case study on a subject suffering from Atopic Dermatitis.

Method of Application: In another preferred embodiment, the invention provides methods of application of the instant compositions. According to this method, the topical composition can be co-administered alongside oral therapy to maximize relief especially in cases of repeated allergen exposure topically, by the following methods of application. The method includes application of an effective amount of the preferred embodiment on the exact location or area around the location of the allergen or irritant exposure; and massaging the preferred embodiment of the invention on the skin, in case of cremes, salves, ointments, lotions, oils or gels.

Proliferative dermatological conditions according to the invention include Psoriasis, Atopic Dermatitis, Dermatitis, and Eczema, includes, but is not limited to other proliferative dermatological disorders such as psoriasis, vitiligo, bullous pemphigoid, actinic keratosis, and any other skin disorders. Atopic dermatitis is a long-term (chronic) skin disorder that involves scaly and itchy rashes, which is due to a hypersensitivity reaction (similar to an allergy) in the skin that leads to longterm swelling and redness (inflammation) of the skin. People with atopic dermatitis may lack certain proteins in the skin, which leads to greater sensitivity. Atopic dermatitis is most common in infants. It may start as early as age 2 to 6 months. Many people outgrow it by early adulthood. People with atopic dermatitis often have asthma or seasonal allergies. There is often a family history of allergic conditions such as asthma, hay fever, or eczema. People with atopic dermatitis often test positive to allergy skin tests. Although, atopic dermatitis is not caused by allergies, however, the condition tends to get worse when the person is exposed to certain triggers. Contact dermatitis is a condition in which the skin becomes red, sore, or inflamed after direct contact with a substance. There are two kinds of contact dermatitis viz., irritant or allergic.

According to another preferred embodiment, the invention provides method for treating and for management/remission of proliferative dermatological disorders comprising administering to a subject in need thereof, a composition comprising a first therapeutic herbal extract standardized for Acetyl keto beta boswellic acid in combination with second therapeutic agent which comprises of a synergistic herbal extract standardized for Bakuchiol with total furanocoumarin content less than 100 ppm isolated from Psoralea corylifolia

In yet another preferred embodiment, the invention provides method for treating and for management/remission of proliferative dermatological disorders comprising administering to a subject in need thereof, a composition comprising a first therapeutic herbal extract standardized for Acetyl keto beta boswellic acid in combination with second therapeutic agent which comprises of a synergistic herbal extract standardized for Bakuchiol with total furanocoumarin content less than 100 ppm isolated from Psoralea corylifolia optionally in association with one or more excipients. The subject is human according to the method of invention.

The method of administrations according to the invention may be oral or topical administration. The topical dosage form however does not alleviate the symptoms of the disease to the same extent as done by the oral dosage four. The most preferred dosage form is therefore oral.

The invention further provides use of composition comprising a first therapeutic herbal extract standardized to Acetyl keto beta boswellic acid (minimum 20% assay) in combination with second therapeutic agent which comprises of a synergistic herbal extract standardized for Bakuchiol with total furanocoumarin content less than 100 ppm isolated from Psoralea corylifolia optionally in association with one or more excipients, for relief and remission of proliferative dermatological conditions in a subject. The subject is human.

According to the invention, the synergistic composition comprises Bakuchiol (0.1-99.9% w/w) with total furanocoumarin content less than 100 ppm alone, and in combination with a second therapeutic agent which comprises of a synergistic herbal extract standardized for high assay of 3-o-Acetyl-11-keto-beta-boswellic acid (BOSWELLIC ACID) (20.00-90% w/w). The combination of these two therapeutic agents in a preferred composition provides for equivalent relief and remission of proliferative dermatological conditions as is obtained by standard steroidal therapy consisting of but not limited to methyl prednisolone, halobetasol in combination with immunosuppressant drugs like thioazaprine.

The composition according to the invention provides superior remission of dermatological conditions as detailed herein below when compared to existing and/or single anti-inflammatory compounds derived from herbal extracts. Specifically, the combination is highly effective in treatment/relief of various skin proliferative disorders, specifically mild to moderate Psoriasis, providing excellent remission in humans. This combination is also of use in other proliferative disorders such as Atopic dermatitis, Dermatitis, Eczema, Bullous Pemphigoid, and the like.

According to another preferred embodiment, the compositions of the present invention optionally comprise other therapeutic agent including crude herbs, herbal powder, standardized herbal extracts, or Phyto constituents (high or low purity) thereof, including, but not limited to Kalmegh (Andrographis paniculata Nees), Guduchi (Tinospora cordifolia), Alfalfa (Medicago Saliva), Andrographolide (Andrographis Paniculata), Glycyrhizzic acid (Glycyrhizza Glabra), Angelica (Angelica Archangelica), Arnica, Black Cohosh (CimicifugaRacemosa), Boswellia serrata, Cayenne Pepper, Celery (ApiumGraveolens), Ginger (ZingiberOfficinale), Ginseng (PanaxQuinquefolius), Hops (HumulusLupulus), Licorice extract (Glycyrrhizaglabra), Mustard Plaster (Brassica Alba), Oregano (OriganumVulgare), Pineapple, Rosemary (RosemarinusOfficinalis), Sesame Seeds (Sesamumlndicum), Stinging Nettle, Turmeric (Curcuma Longa), Willow (Salix), Wintergreen (GaulteriaProcumbens) and the likes.

The compositions are formulated in a suitable vehicle comprising of pharmaceutical inert ingredients, excipients, diluents, binders, fillers, preservatives, emollients, penetrating enhancers, solubilizers, moisturizers, such as but not limited to cyclodextrin, methyl cellulose, paraffins, castor oil, almond oil, vitamin E, methyl paraben, propyl paraben and the like, into an oral dosage form such as tablet, softgel, or capsule, or optionally as a topical dosage form such as ointment, gels, emulgels, nanoemulgels, lotions, creams and the like, by using the methods known in the art.

Although, 3-o-Acetyl-11-keto-beta-boswellic acid (BOSWELLIC ACID) and Bakuchiol are known as anti-inflammatory compounds, but none of the prior art reports their specific compositions, mentioned herein, along with usage and efficacy of highly pure standardized compositions in humans as demonstrated in the instant invention. While 3-o-Acetyl-11-keto-beta-boswellic acid (BOSWELLIC ACID) alone itself provides substantial remission, unexpected excellent synergestic efficacy was found in the combination of 3-o-Acetyl-11-keto-beta-boswellic acid (BOSWELLIC ACID), in combination with low dosages of Bakuchiol, which provided complete remission without any side effects, and no relapse for extended periods of time.

While the two compounds—Bakuchiol and Boswellic acid standardized to 3-o-Acetyl-11-keto-beta-boswellic acid (BOSWELLIC ACID) separately show an IC₅₀ value of 3.1 um and 1.7 um in a LOX inhibition assay, the combination showed a highly potent IC₅₀ of 0.5 um for the same concentration in LOX inhibition assay

### EXAMPLES

### Example 1

### Method for Quantification of Bakuchiol and Detection of Presence of Furanocoumarins at 100 ppm Levels

1. Chromatographic Conditions:—

a) Suitable High Performance Liquid Chromatograph equipped with anisocratic pump.
b) Waters 2489 UV/Vis. detector & and Integrator or suitable chromatographic Software
c) Column: Waters Spherisorb 5 micron 4.6×250 mm

### 2. Reagent:

a) HPLC grade water
b) Acetonitrile (HPLC grade)
c) Methanol (HPLC grade)

### 3. Mobile Phase:—Water: Acetonitrile::30:70

### 4. Chromatographic Parameter:—

### Detection: 265 nm for Bakuchiol

### 245 nm for Psoralen&Isopsoralen

### Injection Volume: 20 μl

Flow Rate: 1.0 ml/min
Sample concentration: 0.1 mg/ml

### Example 2

### Extraction of Crude Babchi from Psoralea Plants

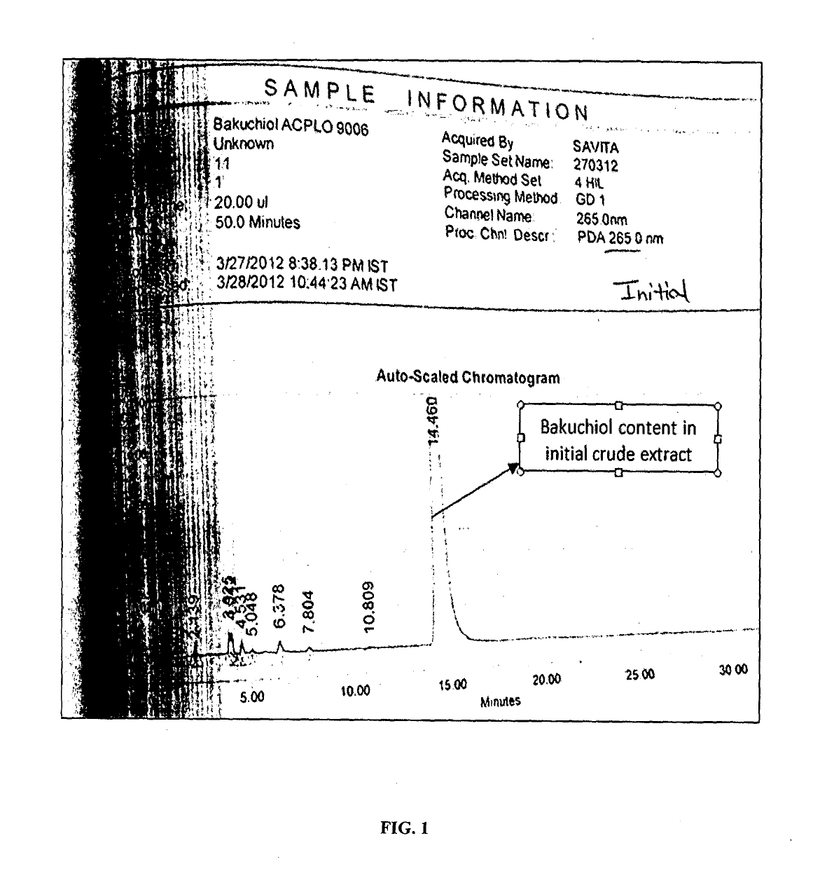

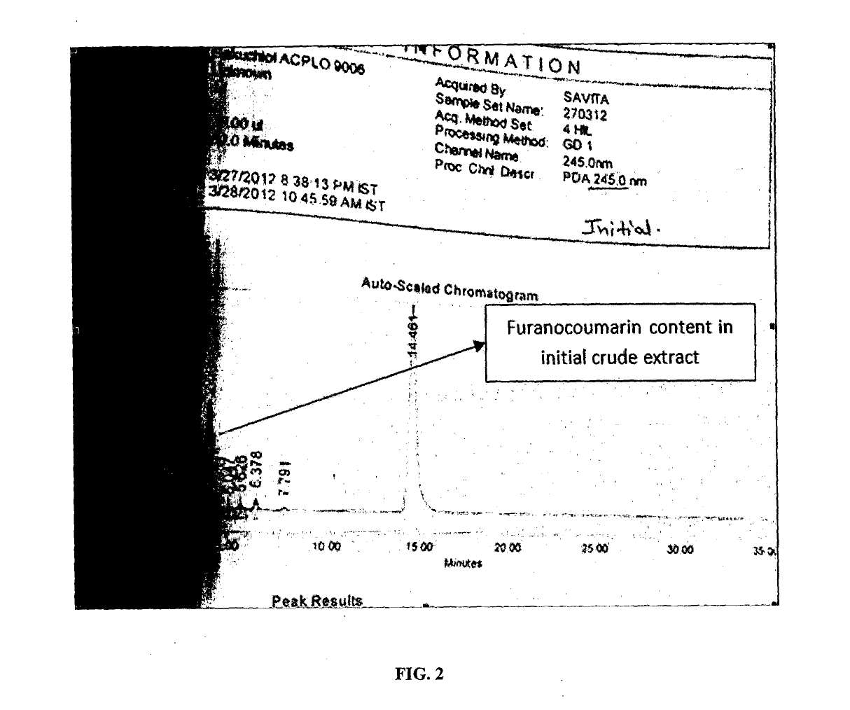

In a 50 litre flask, Hexane 15 litre and Psoralea corylifolia plant seed powder, 4 kg were charged and the mixture was mixed in an agitator at room temperature then slowly the temperature was raised up to reflux in water bath for three hours. The hot mixture was filtered through a filtrate that was collected. To the retention cake, the same process was repeated three times with fresh solvent. All collected filtrate was combined, solvent was removed through evaporator and the residue was dried under high vacuum distillation to yield 0.8 kg of crude extract having 50-55 gm of Babchi. The extract was analyzed by HPLC analysis, as” shown as Initial Psoralea corylifolia extracts at 265 nm, 245 nm in FIGS. 1 and 2 respectively.

### Example 3

### Purification of Bakuchiol from Crude Extract

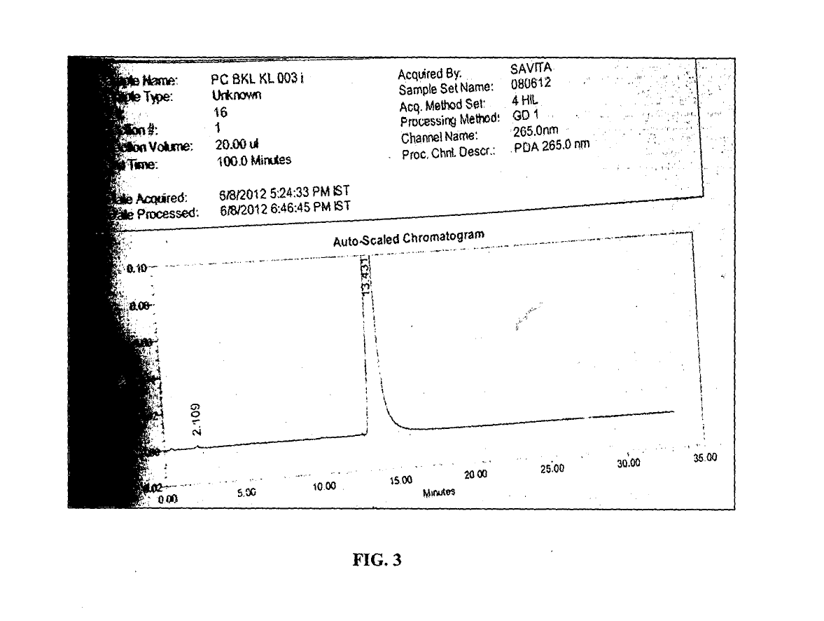

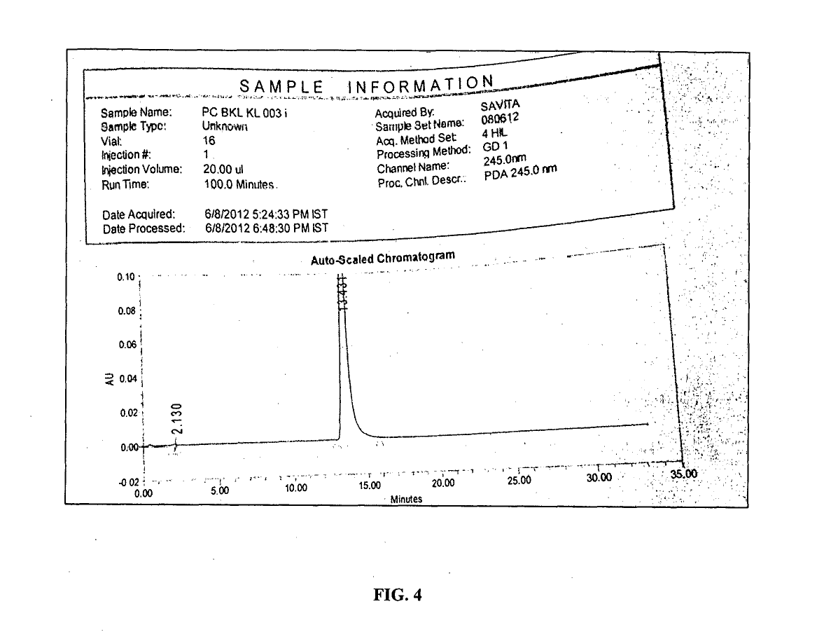

0.8 kg crude Babchi material was dissolved in methanol and loaded into prepared silica column 6×36 cm. The column containing flash silica 10 kg in petroleum ether solvent equilibrated with four column volume of petroleum ether. The loading material was fed at top of the column and eluted with 10 litre of petroleum ether, followed by 5 liter petroleum ether and ethyl acetate (10:0.05), 10 litre petroleum ether and ethyl acetate (10:0.1), and 10 lit petroleum ether and ethyl acetate (10:0.2), to elute Babchi. The eluent was monitored by Thin Layer Chromatography (TLC) till Babchi was completely eluted from the column. Fractions containing only Babchi were combined and the solvent was evaporated under vacuum. Using this method highly pure Bakuchiol, 150 gm was obtained. Earlier and later fraction were also combined and evaporated the solvent to obtain less pure Babchi, which is further purified by column chromatography to obtain pure 50 gm Bakuchiol as shown as High purity extract of Psoralea corylifolia, standardized to Bakuchiol at 265 nm, 245 nm in FIGS. 3, 4 respectively. Alternately, a semi or completely chemically synthesized Bakuchiol may be used in the instant formulations.

### Example 4

### Method for Quantification of 3-o-Acetyl-11-Keto p-Boswellic Acid (BOSWELLIC ACID) by HPLC

### Column: Kromasil C-18

### Wavelength: 247 nm

Flow: 1.5 ml/min
Mobile phase: Methanol: Buffer: 90:10
Buffer details: 95 ml H2O: 5 ml ACN, adjust pH 2.8 with phosphoric acid
Sample preparation: 2 mg/ml

### Example 5

### Extraction of 3-o-Acetyl-11-keto p-boswellic acid (BOSWELLIC ACID) from Boswellia Serrata

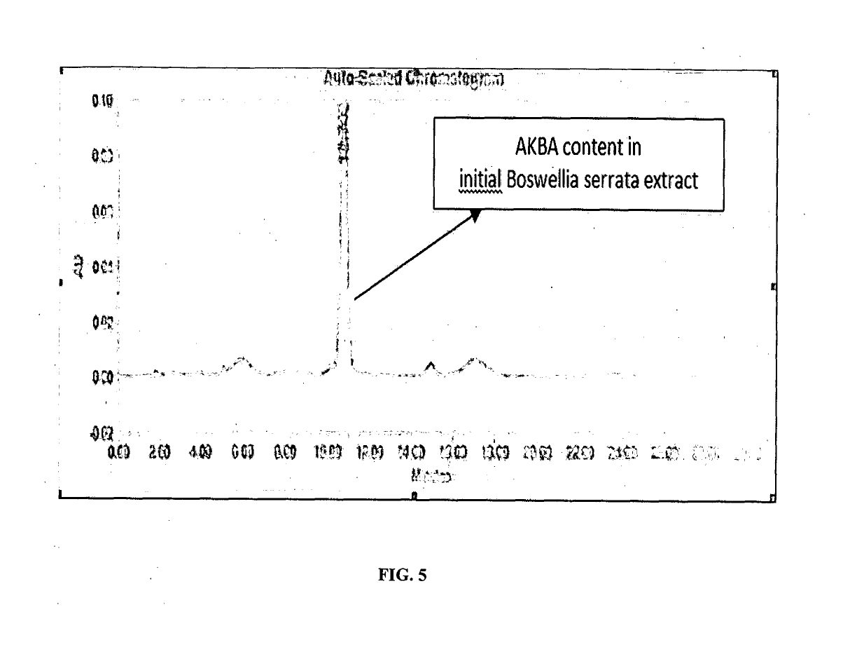

1 Kg crushed powder of Boswellia serrata gum resin was extracted with methanol 3.0 liter at 40-50° C. The extraction process was repeated twice with Methanol, 3.0 liter and residue was discarded. Total methanol extract after filtration was concentrated in vacuum to obtain dark brown syrupy mass of 900 gm. The syrupy mass was dissolved in sodium hydroxide solution, to which Methylene chloride, 1.0 liter was added and the by-product was extracted. The basic layer was acidified with dilute Hydrochloric acid, at pH 3-4. All the boswellic acids that precipitate out were filtered and dried at 50-60° C. in a vacuum dryer. A cream to yellowish powder was obtained which contained about 20% BOSWELLIC ACID. The extract was analyzed by HPLC analysis, as shown as initial Boswellia serrata extract, standardized to BOSWELLIC ACID, in FIG. 5.

### Example 6

### Purification of 3-o-Acety11-keto p-boswellic acid (BOSWELLIC ACID) from Crude Extract

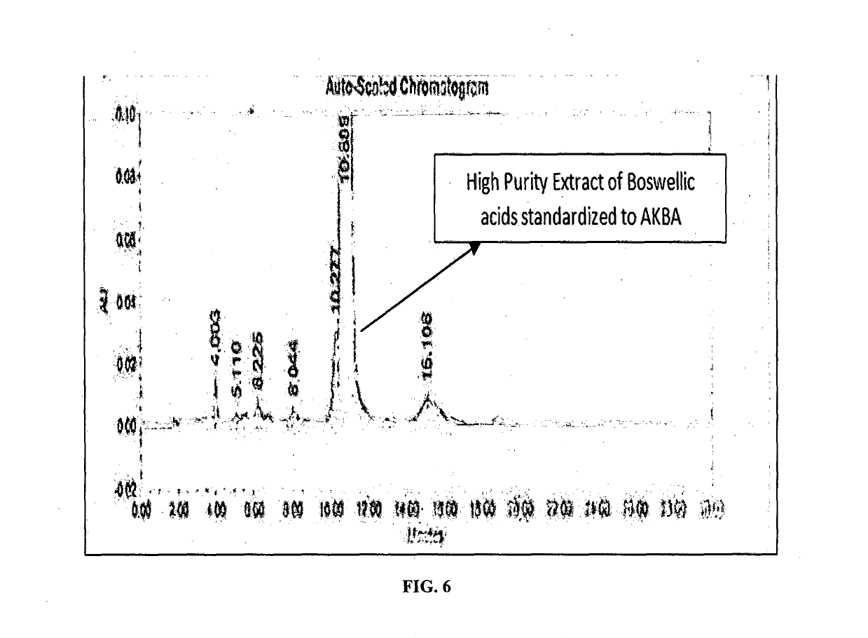

10 g of crude mass of BOSWELLIC ACID was purified by subjecting the crude to column chromatography on silica column using 200 ml Hexane; 200 ml Ethyl acetate/hexane; and 200 ml 12 percent Ethyl acetate/hexane as elutes. Each 20 ml fraction was collected. Fractions containing BOSWELLIC ACID were collected and combined and evaporated to obtain 1.00 g residue which contained BOSWELLIC ACID (80% pure by HPLC), that further purified by repeated column chromatography to obtain 3-o-Acetyl-11-keto beta boswellic acid (Assay by HPLC 95%). This was analyzed by HPLC analysis, as shown in FIG. 6.

Alternately, a semi or completely chemically synthesized Acetyl-11-keto p-boswellic acid may be used in the instant formulations.

### Example 7

### Testing of Externally Sourced Capsaicin

Capsaicin was sourced from a manufacturer in South India, and tested in-house by HPLC, under following conditions:

### LC Conditions:

### Column: MD-150 (3×150 mm; 3 μm)

Mobile Phase: 50 mM Ammonium Acetate, pH 4.4 with acetic acid; 45% Acetonitrile; Flow Rate: 0.8 mL/min; Temperature: Ambient; Injection Volume: 20 μL
Detectors and Conditions: Electrochemical Detector: Model 5600A, CoulArray with Model 5010 AnalyticalCell.
Applied Potentials: +150, +450 mV vs. Pd.

### UV Detector: Model(s) 520 (or 522)

### Wavelength: 235 nm and 280 nm

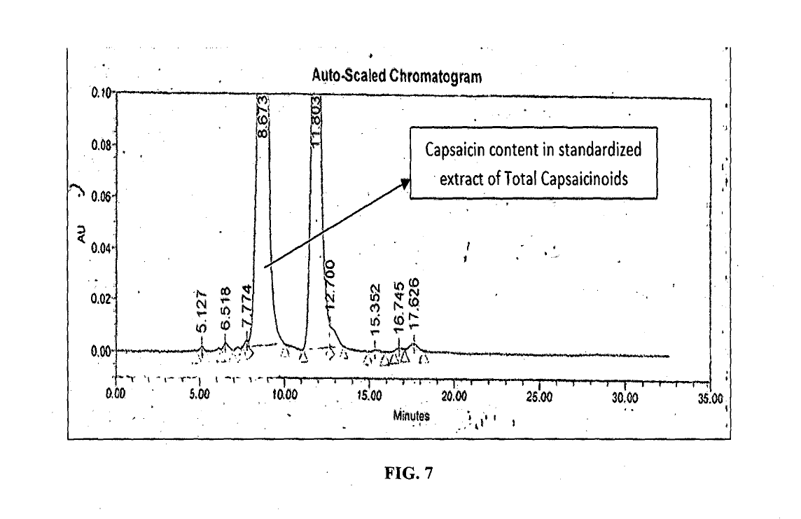

This was analyzed by HPLC analysis, as shown in FIG. 7.

### Example 8

### Compositions for Treatment/Management of Pain

TABLE 6

| | | Composition(% w/w) | | | |
|---|---|---|---|---|---|
| | | Formu- | Formu- | Formu- | Formu- |
| Sr. | | lation | lation | lation | lation |
| No. | Ingredients | A | B | C | D |
| 1 | Bakuchiol | 2 | 1.5 | 2.5 | 4 |
| 2 | 3-o-Acetyl-11- | — | — | 1.0 | — |
| | keto beta | | | | |
| | boswellic acid | | | | |
| 3 | Capsaicin | — | — | — | 0.5 |
| 4 | Glycerine | 6 | 6 | 4 | 5 |
| 5 | Water | 48 | 48 | 50 | 50 |
| 6 | Cetyl Alcohol | — | 2 | — | 1.50 |
| 7 | Cetostrearyl | — | — | — | 1.50 |
| | alcohol | | | | |
| 8 | Emulsifying | 7 | 6 | 5 | 10 |
| | wax | | | | |
| 9 | Jojoba oil | 10 | 12 | 5 | 10 |
| 10 | Castor oil | — | — | 10 | 5 |
| 11 | Almond oil | — | — | 1.8 | — |
| 12 | Cedarwood oil | — | — | — | 2.00 |
| 13 | Vitamin E Oil | — | 5 | — | — |
| 14 | Methyl | 2 | 2 | — | — |
| | sulfonyl | | | | |
| | methane | | | | |
| 15 | Wintergreen oil | — | — | 2 | — |
| 16 | dimethicone | — | — | 2 | 2.2 |
| 17 | Methyl paraben | — | — | 0.1 | 0.1 |
| 18 | Propyl paraben | — | — | 0.1 | 0.1 |
| 19 | Glyceryl mono | 6 | 7 | 8.50 | — |
| | stearate | | | | |
| 20 | Eucalyptus oil | 4 | 3 | — | — |
| 21 | Boswellia oil | — | 4.90 | 8 | 0.1 |
| 22 | Wheatgerm oil | 10 | 2.6 | — | 8 |
| 23 | Lavender oil | 5 | — | — | |

### Example 9

### Compositions for Treatment/Management of Pain

TABLE 7

| | | | | Formu- | |
|---|---|---|---|---|---|
| | | | | lation | |
| | | Formu- | Formu- | D | Formu- |
| | Formu- | lation | lation | (F-14) | lation |
| | lation | B | C | Not part of | E |
| Ingredients | A | (F-32) | (F 21) | Invention | (15 C) |
| Rose Water | | | 48.98 | 50.55 | 50.35 |
| Water | 51 | 49 | | — | |
| Glycerein | 5 | 4.8 | 3.91 | 3.8 | 5.05 |
| Jojoba oil | 5 | 11 | 9.78 | 10.47 | 10.1 |
| Castor Oil | 3.0 | 4 | 2.93 | 3.8 | 3.79 |
| Cedar wood | 2.0 | 2 | 2.93 | 1.9 | 2.94 |
| Boswelia oil | 2.5 | 2.5 | 2.93 | 2.8 | 2.94 |
| Vitamin E | 1 | 3 | 3.91 | 3.8 | 3.79 |
| Eucalyptus oil | 1 | 3 | 2.88 | 4.2 | 3.79 |
| Almond oil | 5 | | | | |
| Emulsifying | 7.9 | 5.5 | 6.84 | 6.19 | 5.05 |
| wax | | | | | |
| GMS | 8.5 | 8.5 | 9.78 | 9.5 | 9.52 |
| Cetyl Alcohol | 1.5 | 0.75 | | | |
| Cetostearyl | 1.5 | 0.75 | | | |
| alcohol | | | | | |
| Dimethicone | 1 | 1 | | | |
| Bakuchiol | 1.9 | 2 | 2.93 | 2.8 | 2.52 |
| Methyl and | 0.2 | 0.20 | 0.19 | 0.19 | 0.16 |
| Propyl paraben | | | | | |
| Methyl | 2 | 2 | 2.00 | | |
| sulphonyl | | | | | |
| methane | | | | | |

### Study on Topical Compositions

Homogenieity—Homogenieity and lustre/lightness were tracked by visual inspection and subjective notes. Homogeneity is scored on a scale of 1-3, with 1 being barely homogenous, and 3 for highly homogenous. Lustre/lightweight nature is scored on a scale of 1-3, with 1 being not lustrous, and 3 for lustrous.

pH measurement—pH measurement was carried out as per procedures outlined in the USP (1 g of cream/100 ml of DM water).
Viscosity testing—A digital viscometer was used for measurement of viscosity, all at N.T.P
Spreadability—Spreadability of the cream was calculated as per the formula, S=M×L/T, where M is weight of the upper glass slide apparatus, L is the length of the glass slide, and T is the time taken for separation of both the slides.
In-vitro drug diffusion—In-vitro drug diffusion data for optimizing formulations was carried out on a modified Franz diffusion cell apparatus (10 ml compartment capacity). Suitable membrane was placed between the two compartments. The cream formulation sample was placed on top of membrane and was further filled with buffer solution in the physiologically compatible pH range of 7.2-7.4, and maintained under stirring at 60 rpm and physiological body temperature of 37.2±0.5° C. Sample were withdrawn at 10 minutes, 30 minutes, 60 minutes, 120 minutes, 180 minutes, 240 minutes, and 480 minutes were analysed on HPLC for diffused drug quantity.

TABLE 8
Diffusion data on various batches

| | Bakuchiol | | |
|---|---|---|---|
| Batch | Content | Diffusion | Comment |
| F14 | 99.10% ± 0.4 | 54.34 ± 1.2 | Assay of Bakuchiol |
| (Example 9 | | | is < than 50% |
| Formu- | | | |
| lation D) | | | |
| F15 C | 99.28% ± 0.2 | 78.26 ± 1.72 | Assay of Bakuchiol |
| (Example | | | is > than 50% |
| 9 - E) | | | |
| F21 | 99.56% ± 0.1 | 85.33 ± 1.19 | Assay of Bakuchiol above |
| (Example 9 | | | 50% and Combination of |
| Formu- | | | penetration enhancers |
| lation C) | | | further enhances diffusion |

Conclusion from Diffusion Studies
1. Assay of Bakuchiol should be greater than 50%
2. Combination of penetration enhancers provides higher diffusion
3. Diffusion after 8 hrs should be greater than 60%, preferably greater than 70% and most preferably greater than 80%.
   Drug Content—Drug Content was carried out by taking a fixed quantity of cream, dissolving it in methanol (100 mg in 1000 ml), and injecting this diluted sample directly into the HPLC for quantification by HPLC against standard sample using a validated procedure.
   Stability studies—A concurrent stability data check for optimized formulations to check for formulation stability at accelerated stability conditions of (40±2)° C., 75±5% Relative Humidity in primary laminated aluminium tube was carried out as per USP guidelines. Following is the compilation of data on initial and stability samples.

TABLE 9
Stability data

| | | | | | | | Drug |
|---|---|---|---|---|---|---|---|
| Time | | Homogeneity | | | Spreadability | Viscosity | Content |
| (days) | Appearance | Score | pH | Lustre | (g · cm/sec) | (cps) | (% w/w) |
| 0 | Initial data | 3 | 7.4 | High | 29.32 ± 0.21 | 15558 ± 17 | 99.63% ± 0.2 |
| 15 | no change | 3 | 7.4 | High | 29.66 ± 0.09 | 15586 ± 13 | 99.60% ± 0.1 |
| 30 | no change | 3 | 7.3 | High | 28.87 ± 0.39 | 15498 ± 24 | 99.58% ± 0.2 |
| 45 | no change | 3 | 7.2 | High | 29.14 ± 0.22 | 15526 ± 19 | 99.61% ± 0.1 |
| 60 | no change | 3 | 7.4 | High | 29.41 ± 0.32 | 15581 ± 24 | 99.66% ± 0.2 |
| 90 | no change | 3 | 7.4 | High | 29.36 ± 0.15 | 15456 ± 15 | 99.57% ± 0.2 |

Conclusion: Initial and stability data is satisfactory. No significant change on stability in any parameter tested indicates stable composition.

### Compositions for Psoriasis

### Example 10

### Composition of Oral Capsule Comprising Boswellic Acid and Bakuchiol

TABLE 10

| | | Per capsule |
|---|---|---|
| Ingredient Name | Specification | (mg) |
| Boswellic acid | Boswellic acid | 225 |
| standardized to | standardized to 39% of 3- | |
| 3-o-Acetyl-11-keto beta | o-Acetyl-11-keto beta | |
| boswellic acid | boswellic acid | |
| Bakuchiol | Assay: 70% by HPLC | 50 |
| EXP (Yellow dextrin)/ | | 65 |

Process: Mix Boswellic acid, Bakuchiol and yellow dextrin for 15 minutes and fill in the capsule.
Dose: Two capsules at the breakfast and two at dinner.
Result: Lesion progression is halted however improvement is slow. It further necessitates use of ingredients having higher content/assay values.

### Example 11

### Composition of Oral Capsule Comprising Boswellic Acid and Bakuchiol

TABLE 11

| S. | | | Per capsule | % |
|---|---|---|---|---|
| No | Ingredient Name | Specification | (mg) | composition |
| 1 | Boswellic acid | Boswellic acid | 175 | 43.75 |
| | standardized to 3-o- | standardized to | | |
| | Acetyl-11-keto beta | 44% of 3-o- | | |
| | boswellic acid | Acetyl-11-keto | | |
| | | beta boswellic | | |
| | | acid | | |
| 2 | Bakuchiol | Assay: 92% by | 25 | 6.25 |
| | | HPLC | | |
| 3 | EXP (Yellow | | 200 | 50 |
| | dextrin) | | | |

| | | |
|---|---|---|
| Total | 400 | 100 |

Process: Boswellic acid, Bakuchiol and yellow dextrin for 15 minutes and fill in the capsule.
Dose: Two capsules at the breakfast and two at dinner.
Result: Successful relief and management of Psoriasis (Inverse, Pustular, Plaque, Palmoplantar)
Conclusion from example 10 and 11: Higher assay of Boswellic acid standardized to 3-o-Acetyl-11-keto beta boswellic acid and higher assay of Bakuchiol provide better control over psoriasis.

### Compositions for Dermatitis

### Example 12

### Composition of Oral Capsule Comprising Boswellic Acid and Bakuchiol

TABLE 12

| S. | Ingredient | | Per capsule | Per capsule |
|---|---|---|---|---|
| No | Name | Specification | (mg) | (mg) |
| 1 | Boswellic acid | Boswellic acid | 50 | 100 |
| | | standardized to 33% of | | |
| | | 3-o-Acetyl1-keto p- | | |
| | | boswellic acid | | |
| 2 | Bakuchiol | Assay: 92% by HPLC | 15 | 10 |
| 3 | EXP (Yellow | | 185 | 10 |
| | dextrin) | | | |

| | | |
|---|---|---|
| Total | 250 | 120 |

Process: Mix Boswellic acid, Bakuchiol and yellow dextrin for 15 minutes and fill in the capsule.
Dose: Two capsules at the breakfast and two at dinner.
Result: Successful relief and substitution of steroidal therapy in case of adult Atopic dermatitis
Conclusion: A therapeutically effective range for Boswellic acid content between 30-70 mg in combination with 10-50 mg Bakuchiol is a preferred and innovative combination as it provides complete relief

### Tablet Composition for Dermatitis

### Example 13

TABLE 13

| S. | | | Per tablet |
|---|---|---|---|
| No. | Name | Percentage | (mg) |
| 1 | Boswellic acid standardized | 28.57 | 100 |
| | to 45% of 3-o-Acetyl-ll-keto | | |
| | p-Boswellic acid | | |
| 2 | Baluchiol | 14.28 | 50 |
| 3 | Lactose | 39.14 | 137 |
| 4 | Corn Starch | 8.57 | 30 |
| 5 | Magnesium Stearate | 0.85 | 3 |
| 6 | HydroxyPropoyl cellulose | 8.57 | 30 |
| | Total | 100 | 350 |

### Process

1. Blend actives Boswellic acid with BOSWELLIC ACID (45% w/w) and Bakuchiol with lactose, corn starch and HydroxyPropoyl cellulose.
2. Blend the mixture of step 1 with magnesium stearate
3. Compress the blend of step 2 into tablets.

### Example 14

### Syrup for Dermatitis

TABLE 14

| S. | | | Batch size |
|---|---|---|---|
| No. | Name | Percentage | in g |
| 1 | Boswellic acid standardized to 45% of | 4 | 100 |
| | 3-o-Acetyl-ll-keto p-Boswellic acid | | |
| 2 | Bakuchiol | 0.4 | 10 |
| 3 | Benzoic Acid | 0.012 | 0.3 |
| 4 | Potassium Sorbate | 0.004 | 0.1 |
| 5 | Propylene Glycol | 8 | 200 |
| 6 | Sucralose | 30.4 | 760 |
| 7 | Purified Water | 57.184 | 1429.6 |
| | Total | 100 | 2500 |

Per serving i.e. per 2.5 ml-100 mg of BOSWELLIC ACID and 10 mg of Bakuchiol.

### Process:

1. Dissolve 100 g BOSWELLIC ACID and 10 g Bakuchiol in 500 g water
2. Add benzoic acid, potassium sorbate and dissolve
3. Add propylene glycol and stir
4. Add sucralose and dissolve
5. Make up the volume with remaining quantity of water and filter.

### Example 15

### Oral Capsule for Psoriasis

TABLE 15

| S. | | Per capsule | Total | % |
|---|---|---|---|---|
| No | Ingredient Name | (mg) | (g) | composition |
| 1 | Boswellic acid standardized to | 224.8 | 5.62 | 51.09 |
| | 44% of 3-o-Acetyl-ll-keto p- | | | |
| | Boswellic acid | | | |
| 2 | Bakuchiol (80% assay) | 62 | 1.55 | 14.09 |
| 3 | Yellow dextrin | 153.2 | 3.83 | 34.81 |

| | | | |
|---|---|---|---|
| Total | 440 | 11.00 | 100 |

Result: A twice a day regimen seems to be the optimal with once in the morning and once in the night, and active ingredient level at which it is minimally effective seems to 150 mg of Boswellic acid equivalent to BOSWELLIC ACID, and 10 mg of Bakuchi oil (PC) daily.

### Example 16

### Safety Profile for Topical Composition

A repeated insult patch test was carried out on the skin of multiple healthy volunteers. Repeat applications of cream on elbows of healthy volunteers were carried out and response checked for parameters such as redness, itching, pruritus, dryness and any other irritation. No appearance of adverse effect was noted as being compatible with multiple skin types. Repeat applications did not result in erythema or edema indicating that test product will not cause any dermal irritation or any allergic sensation.

### Example 17

### Pilot Study for Pain Relief Using Combination of Bakuchiol and Boswellia Oil

### Patient Information:

Patients feedback was collected from a total of 49 patients suffering from acute and chronic musculoskeletal inflammatory disorders (17 users suffering from osteoarthritis, 11 patients suffering from Rheumatoid Arthritis, 4 patients suffering from frozen shoulder, 8 patients suffering from post-traumatic synovitis, and 9 patients suffering from sprains) Patients with clinically active renal, hepatic or peptic ulcer disease, history of alcohol or drug abuse, concomitant skin disease or abrasions at the application site and those patients who were using any other topical product at the application site were excluded from the study.

### Directions for Use:

All users were advised to apply a small quantity of pain relief cream prepared in accordance with example 9 formulation B topically to the affected region, with gentle massage, twice daily for a period of 2 months. All the patients were assessed for joint pain, joint swelling, joint tenderness, joint stiffness/restricted range of motion. Response to pain relief cream was evaluated on a predefined symptom score scale, from 0 to 3 (3=maximum pain and 0=no pain). All the patients were assessed for local adverse reactions like irritation, burning/stinging sensation and erythema

### Result

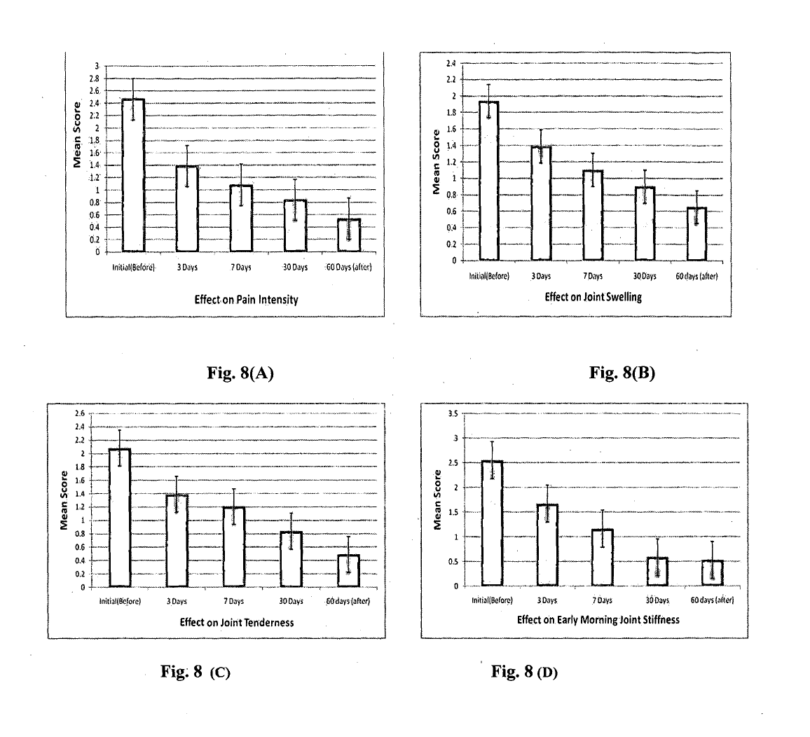

Data collected is presented in FIG. 8/10 and indicates significant reduction (greater than 50% reduction) in pain intensity, joint swelling, joint tenderness and early morning joint stiffness at the end of 4 weeks/30 days and at least 60-75% reduction at the end of 8 weeks/60 days.

### Example 18

### Pilot Study for Psoriasis

An optimized phytotherapy treatment regimen of 22 days, twice a day oral tablets/capsules was created, which led to remission of psoriatic pustules, plaques, and dry scaly skin. This was by replacing the standard of care; corticosteroids like Clobetasol and Halobetasol, and methotrexate

### Patients Information:—

Pilot Studies were carried out in a set of 18 adult patients (Age distribution: 29.56±5.033) suffering from Psoriasis: Plaque Psoriasis (n=10), & Pustularpsoriasis (n=5), & Palmoplantar (nonpustular) psoriasis (n=3) Two patients dropped out during the course of the study due to personal reasons (not relating to treatment). All patients enrolled had a PASI score of below 60. None of the patients in the study taken a prior course of biologics
Patients with clinically active renal, hepatic or peptic ulcer disease, history of alcohol or drug abuse and those patients who were using any other systemic corticosteroids, biologics, or immunosuppressant for a period of 4 weeks from the study, were excluded from the study.

### Directions for Use:

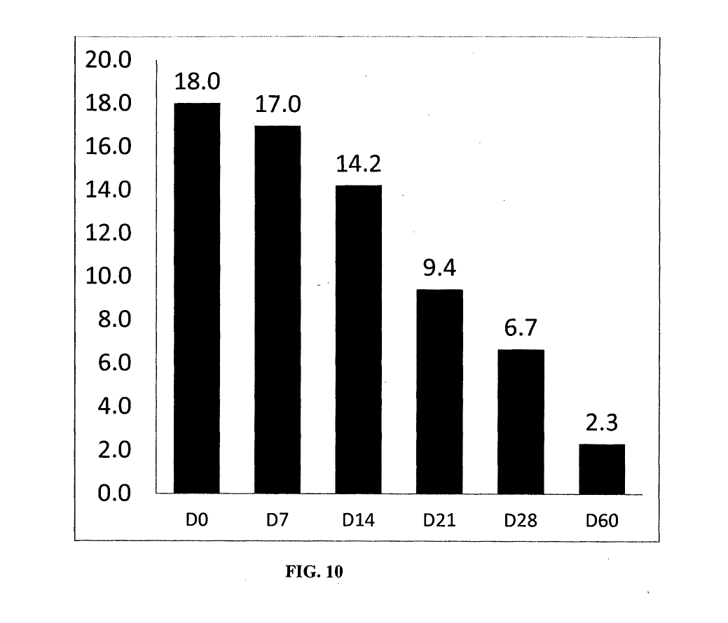

All adult users were advised to take two capsules (400 mg) daily b.i.d. concomitant with their meal. All the patients recorded their scores on the validated PAST (Psoriasis area & severity index) scale, monitored for physical and well as subjective symptoms, on a scale from 0 to 72, refer FIG. 10/10 (reduction of PASI Score).

### Example 19

### Case Studies for Psoriasis

TABLE 16

| | Case 1 | Case 2 | Case 3 |
|---|---|---|---|
| | Plaque Psoriasis | Inverse Psoriasis | Pustular Psoriasis |
| Patient | A 37 year male presents | A 36 year female | A 32 year male |
| Information | with patches of rough, | presents with red | presents with |
| | red skin, with silvery- | scaly lesions under | pus-filled bumps |
| | white scales on both | arm-pits, and in | (pustules) |
| | elbows and knees. | various skin folds. | surrounded by |
| | | | red skin, and |
| | | | frequently |
| | | | occurring fever, |
| | | | chills and nausea. |
| Conventional | Therapy I: Vitamin D | Therapy I: Topical | Therapy I: |
| therapy | preparation | Corticosteroids | Unknown due to |
| and | (Duration: 4 weeks) | (diluted) (1% | non- |
| outcome | Therapy II: Topical | hydrocortisone) | maintainence of |
| | Corticosteroids + | (Duration: 2 weeks) | medical records. |
| | Salicylic Acid | Therapy II: Topical | |
| | (Duration: 6 weeks) | Corticosteroids | |
| | Therapy III: Oral | (diluted) (2% | |
| | Corticosteroids + Coal | hydrocortisone) | |
| | Tar + Overexposure to | (Duration: 3 weeks) | |
| | UV light | Therapy III: | |
| | (Duration: 4 weeks) | Methotrexate + | |
| | Therapy IV: Oral | Topical steroids | |
| | Corticosteroids + | (Duration: 6 weeks) | |
| | Topical corticosteroids + | | |
| | Skin barrier cream | | |
| | (Duration: 16 weeks) | | |
| | Outcome - Therapies I, | | |
| | II, III, IV did not | | |
| | result in any relief. | | |
| Therapy | Therapy V: | Current Therapy | Current |
| according | Phytotherapy X-22 (312 | V: Phytotherapy X- | Therapy I: |
| to present | mg b.i.d.) (Duration: | 25 (313 mg b.i.d.) | Phytotherapy X- |
| invention | 2 weeks) | (Duration: 8 weeks) | 25 (313 mg b.i.d.) |
| and | Current Therapy VI: | Outcome - A | (Duration: 8 |
| outcome | Phytotherapy X-25 (313 | Phytotherapy course | weeks) |
| | mg b.i.d.)(Duration: | of 4 weeks was | Outcome - |
| | 4 weeks) × 2 | started. Remission | Remission and |
| | Outcome - Therapies V | and symptomatic | symptomatic |
| | provided relief but | relief was obtained | relief was |
| | showed relapse within | with Phytotherapy | obtained with |
| | 11 days of after | as standalone | Phytotherapy |
| | medication halted. | medication. A twice | starting from |
| | Therapy VI was settled | daily dose provided | week 3, as a |
| | upon as the optimal | continued | standalone |
| | balance between disease | remission. No minor | medication. |
| | remission and drug | or major side | PASI score |
| | intake duration. | effects, or gastro- | reduced from |
| | | intestinal side | baseline level of |
| | | effects were | 17.0 to 4.5 over a |
| | | experienced. | 8 week period |
| | | PASI score reduced | |
| | | from baseline level | |
| | | of 18.2 to 2.0 over a | |
| | | 8 week period | |

Conclusion: The current mixture of two phytochemicals, namely, Boswellic acid and Bakuchiol, in its preferred embodiment brings about a pronounced reduction in PASI scores and the levels of psoriasis in patients.

### Example 20

### A User Study was Carried Out for Checking Efficacy of Intervention

### Methodology:

Pilot studies were carried out in a set of 09 adult patients suffering from Atopic Dermatitis (n=6) & Dyshidrotic dermatitis (n=3). A Separate Pilot study was carried out in a set of 21 pediatric patients suffering from moderate to severe Atopic Dermatitis. Data has been recorded over a period of 2 months, and has been analyzed for trends. Patient feedback was collected from all 32 users, using a clinically validated PO-SCORAD questionnaire/App and follow up which tracked Atopic Dermatitis parameters as dryness, redness, itching, swelling, crusting thickening and scratch marks, in addition to body area coverage and subjective symptoms. Data has been converted to a PO-SOCRAD score and analyzed for trends.

### Users:

User feedback was collected from a total of 32 patients suffering from moderate to severe Atopic dermatitis (6 adult users suffering from moderate to severe AD, 3 patients suffering from Dyshidrotic Dermatitis, 21 pediatric patients suffering from Atopic dermatitis) Patients with clinically active renal, hepatic or peptic ulcer disease, history of alcohol or drug abuse and those patients who were using any other systemic corticosteroids or immunosuppressants for a period of 4 weeks from the study, were excluded from the study.

### Directions for Use:

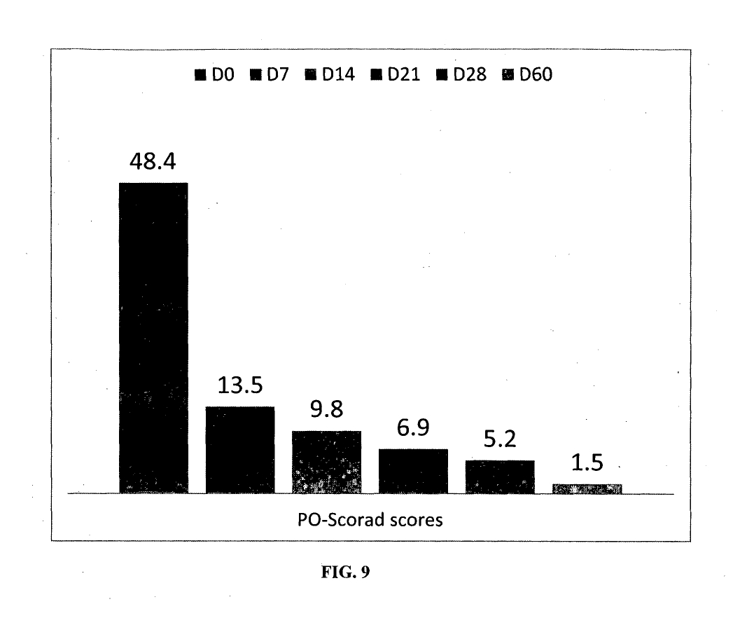

All adult users were advised to take one capsule containing 100 mg of Boswellic acid and 10 mg Bakuchiol (Example 12) daily at night concomitant with their meal. For pediatric patients a once daily suspension of 2.5 ml (Example 14, a small teaspoon was to be given at night concomitant with the meal. All the patients recorded their scores on the validated PO-SCORAD scale, monitored for physical and well as subjective symptoms, on a scale from 0 to 100. Refer FIG. 9 of 10.

### Example 21

### Pilot Study for Dermatitis

### Methodology

A user trial over 60 days with the optimized oral dosage form for Dermatitis showed excellent efficacy and improvement in baseline PO-SCORAD scores, which is a score for the disease index

TABLE 17
Pilot study for Dermatitis

| | D 0 | D 7 | D 14 | D 21 | D 28 | D 60 |
|---|---|---|---|---|---|---|
| Mean PO-SCORAD score | 48.4 | 13.5 | 9.8 | 6.9 | 5.2 | 1.5 |
| StdDev | 13.2 | 4.8 | 3.9 | 2.7 | 2.0 | 1.4 |
| Std Error | 2.5 | 0.9 | 0.7 | 0.5 | 0.4 | 0.3 |
| Confidence Interval | 6.5 | 2.4 | 1.9 | 1.3 | 1.0 | 0.7 |
| Lower CI | 41.8 | 11.1 | 7.8 | 5.6 | 1.9 | 1.4 |
| Upper CI | 54.9 | 15.9 | 11.7 | 8.3 | 2.0 | 1.4 |

| | |
|---|---|
| Annova | F = 2.27 p < 0.0001; HS |

### Conclusion:

A lower dosage and combination of BOSWELLIC ACID and Bakuchiol works well at providing steroid sparing relief in cases of Dermatitis as a once a daily dose. For serious cases the dosing may be increased to twice a day capsules to treat more chronic cases

### Example 22

### Case Studies for Dermatitis

TABLE 18
Case studies for dermititis

| | Case 1 | Case 2 |
|---|---|---|
| | Mild to moderate case | Severe case |
| | of pediatric dermatitis | of atopic dermatitis |
| Patient | A 5 year child presents | A 32 year male presents with itchy |
| Information | with itchy rashes on his | rashes on his fingers, hands, back of |
| | hands, feet, upper torso, | his neck and shoulder. |
| | including elbow folds, | |
| | back of his | |
| | neck and shoulders. | |
| Conventional | Therapy I: Mild topical | Therapy I: Anti-histamines & |
| therapy and | steroids. | tetracycline (Duration: 1 week) |
| outcome | Therapy I provided | Therapy II: Anti-inflammatory |
| | temporary relief, and | medication & Anti-allergies |
| | relapse within 2 days of | (Duration: 2 weeks) |
| | discontinuation of | Therapy III: Phototherapy (7 |
| | application. | sessions. 400J narrow band UV)& |
| | | Methyl Prednisolone (Duration: 8 |
| | | weeks) |
| | | Therapy IV: Azathioprine & |
| | | Methyl-prednisolone (32 mg × 5) |
| | | (Duration: 6 weeks) |
| | | Therapy V: Methyl Prednisolone |
| | | (32 mg × 2) &Halobetasol ointment (3 |
| | | days a week) (Duration 16 weeks) |
| | | Therapies I, II did not result in any |
| | | relief. |
| | | Therapies III, IV did provide |
| | | complete relief, but relapse within 2 |
| | | days of dosage tapering or |
| | | medication |
| | | halting was seen. Hence a case of |
| | | chronic atopic dermatitis is |
| | | suspected. |
| | | Due to chronic and relapsing type |
| | | Atopic Dermatitis, Therapy V was |
| | | settled upon as the optimal balance |
| | | between disease remission and short |
| | | & long term side effects. |
| Therapy | Current Therapy II: | An initial course of 8 days was tried |
| according to | Phytotherapy X-18a | wherein, the therapy was co- |
| present | 1) An initial course of 8 | administered along with Therapy V, |
| invention | days was tried wherein | followed by complete stoppage of |
| and outcome | the Phytotherapy X-18 | therapy V over days 6, 7, 8. No |
| | was co-administered | relapse or flare-up episode occurred |
| | along with Therapy I, | upon stoppage of Therapy V during |
| | followed by complete | concurrent administration of Phyto- |
| | stoppage of therapy I | therapy. Relapse occurred after |
| | over days 6, 7, 8. | stoppage of Phyto-therapy, 2 days |
| | 2) A follow-up | after day 10. |
| | Phytotherapy course of 4 | A follow-up Phytotherapy course of |
| | weeks was started | 4 weeks was started without co- |
| | without co- | administration with Therapy V as |
| | administration with | above. Immediate remission and |
| | Therapy I as above. | symptomatic relief similar to |
| | Immediate remission | Therapy V was obtained with |
| | and symptomatic relief | Phytotherapy as standalone |
| | similar to Therapy I was | medication. A daily once dose |
| | obtained with | provided continued remission. |
| | Phytotherapy X-18, as | Discontinuation of Phytotherapy led |
| | standalone medication. | to relapse in 3-4 days. |
| | A daily once dose | |
| | provided continued | |
| | remission. | |
| | Discontinuation of | |
| | Phytotherapy at end of 4 | |
| | weeks has not led to a | |
| | relapse in over 6 months, | |
| | with only moisturizers | |
| | and no oral or topical | |
| | medication. | |

### Example 23

### Gel Composition for Management or Treatment of Pain

TABLE 19
Gel composition

| S. | | Weight | Quantity |
|---|---|---|---|
| No | Ingredient Name | Percentage | (gm) |
| 1 | Bakuchiol | 2.00% | 2 |
| 2 | Boswellia Oil | 0.00% | 0 |
| 3 | Hydroxy Ethyl Cellulose | 0.90% | 0.9 |
| 4 | Triethanolamine | 0.45% | 0.45 |
| 5 | Dimethyl Sulfoxide | 2.50% | 2.5 |
| 6 | Methyl Paraben | 0.02% | 0.015 |
| 7 | Propylene Glycol | 3.00% | 3 |
| 8 | Distilled Water | 86.00% | 86 |
| 9 | Propyl Paraben | 0.02% | 0.015 |
| 10 | Jojoba | 0.00% | 0 |
| 11 | MSM | 5.12% | 5.12 |

### Process

1) Weigh out accurate quantities of all materials
2) Add Bakuchiol, Boswellia Oil, and propylene glycol to prepare the oil phase (A) at room temperature
3) Add hydroxy ethyl cellulose, DMSO, Methyl paraben, Propyl paraben, and MSM to distilled water to prepare water phase (B) and heat to 45° C. until clear.
4) Mix Oil and Water phase (A+B) at 45 C, and slowly adjust the pH with Triethanolamine to pH 7.4 under constant stirring
5) After stirring for 10 minutes the mixture is allowed to cool to room temperature and a clear pale yellow coloured gel vehicle is obtained.

1.-71. (canceled)

72. A composition for the treatment/management of pain or skin disorders, comprising: from around 1% to around 15% of Bakuchiol by weight and further comprising Boswellia oil or Boswellic acid or combinations thereof and optionally any other therapeutic agent.

73. The composition according to claim 72, wherein the composition is in or al or topical form.

74. The composition according to claim 73, wherein the composition is an oral composition.

75. The composition according, to claim 74, wherein a ratio of Boswellic acid to Bakuchiol is from 30:1 to 1:5.

76. The composition according to claim 75, wherein the ratio of Boswellic acid to Bakuchiol is preferably from 15:1 to 1:2 and most preferably from 12:1 to 2:1.

77. The composition according to claim 74, wherein Boswellic acid has at least 20% of 3-o-Acetyl-11-keto p-Boswellic acid by weight.

78. The composition according to claim 74, wherein total of Bakuchiol and Boswellic acid is from 30-90% of the total composition by weight.

79. The composition according to claim 73, wherein the composition is a topical composition.

80. The composition according to claim 79, wherein the topical composition is a topical cream, comprising one or more ingredients selected from the group consisting of oils, penetration enhancer, emollient, humectant and preservative.

81. An oral composition Inc the treatment/management of skin disorders, comprising from around 10% to around 90% of Boswellic acid by weight and optionally from 1-50% of Bakuchiol by weight.

82. The composition according to claim 81, wherein Boswellic acid is standardized to at least 20% preferably at least 30% and most preferably at least 40% of 3-O-Acetyl-11-keto p-Boswellic acid by weight.

83. A process for preparing oral composition to treat skin disorders, comprising of Boswellic acid and optionally Bakuchiol, comprising mixing of the Boswellic acid and/or Bakuchiol with one or more of pharmaceutically acceptable excipient.

84. The process according to claim 83, wherein a ratio of Boswellic acid and Bakuchiol is from around 30:1 to around 1:5.

85. The process according to claim 84, wherein the pharmaceutically acceptable excipient is selected from a group consisting of yellow dextrin, cyclodextrin, lactose, microcrystalline cellulose, mannitol, sorbitol, magnesium carbonate, hydroxyl propyl cellulose, hydroxypropylmethylcellulose, polyvinyl pyrrolidones, gum acacia, guar gum, tragacanth, sodium alginate, corn starch, bentonite, sodium starch glycolate, croscarmellose sodium, crospovidone, magnesium stearate, calcium stearate, colloidal silicon dioxide, sodium stearylfumarate, talc, sodium lauryl sulphate, sweeteners, flavors, taste masking agents, viscosity building agents, antioxidants, preservatives, buffers and acids.

86. A method of treating atopic dermatitis and dermatitis, comprising administering, a composition, comprising from 1-90% Boswellic acid by weight and optionally 1-50% of Bakuchiol by weight, wherein at least 50 mg of Boswellic acid and optionally at least 10 mg of Bakuchiol is administered daily.

87. The method of treating atopic dermatitis and dermatitis of claim 86, wherein the composition further comprising from 1-50% of Bakuchiol by weight and at least 10 mg of Bakuchiol is administered daily.

88. A method of treating psoriasis, comprising administering on a skin with psoriasis a composition, comprising from 1-90% Boswellic acid by weight and optionally 1-50% of Bakuchiol by weight, wherein at least 100 mg of Boswellic acid and optionally at least 25 mg of Bakuchiol is administered daily.

89. The method of treating psoriasis of claim 88, wherein the composition further comprising from 50% of Bakuchiol by weight and at least 25 mg of Bakuchiol is administered daily.

90. A treatment of skin disorders that includes treatment of Psoriasis, Atopic Dermatitis, Dermatitis, Eczema, pruritis, vitiligo, bullous pemphigoid, actinic keratosis, itching and any other skin disorders by administering oral composition of claim 81.
